SEQUENCE LISTING (27 sequences)

SEQ ID NO: 1 - DNA, 791 bp, Ovis aries
  CDS (36)..(524)
gagtcattcctgttgcagaagtctacctggacagcatgcaaagcgagagtggtcacgaaggcgatgaagaactggccttcagggacacccagcctccctacataaaccttcctattcccttcacggggaggatcgaagggggtctccaggatggacacaaggtcaccgtcatggggcgcgttctttccacagacgaaaacaggtttcaagtggactttcagatgggcatcagtgacacaaacaacgttgccttccacttcaaccctcggtttgaaggaagcgggtatgtggtctgtaacacgatgcagctaggaaactgggggccggaggagaagaagatgcagatgcccttccagaaggggagtctgtttgagatctgcttcaaggtagacagctcctcgttcaaggtgacggtgaacgggagcatcttcctggattatgcacaccgtttgcccttcgaacaagtcaacgccatctccatcggtggctgtgtgcatgtgtcctatatcagcttccagatatagaaacccctaccgtgtggaagatgttcactgcatggtgctcacatggtcttagaactcagaagcttgatgatattggctcccgattacctcatcaccaaccaatcagaagacatccacgagctgatcacacacccctcaccctctccctcgccctgtgtttaagaccatttcccggaaagtcatcgtggaatttggggtttttaagcttgcctgggctccttgcttggtacctgcaataaatgctgcgctttccttcatccaaaaaaa

SEQ ID NO: 2 - protein, 162 aa, Ovis aries
MQSESGHEGDEELAFRDTQPPYINLPIPFTGRIEGGLQDGHKVTVMGRVLSTDENRFQVDFQMGISDTNNVAFHFNPRFEGSGYVVCNTMQLGNWGPEEKKMQMPFQKGSLFEICFKVDSSSFKVTVNGSIFLDYAHRLPFEQVNAISIGGCVHVSYISFQI

SEQ ID NO: 3 - protein, 162 aa, Ovis aries
MQSESGHEGDEELAFRDTQPPYINLPIPFTGRIEGGLQDGHKVTVMGRVLSTDENRFQVDFQMGISDTNNVAFHFNPRFEGSGYVVCNTMQLGNWGPEEKKMQMPFQKGSLFEICFKVDSSSFKVTVNGSIFLDYAHRLPFEQVNAIFIGGCVHVSYISFQI

SEQ ID NO: 4 - protein, 160 aa, Homo sapiens
MAFSGSQAPYLSPAVPFSGTIQGGLQDGLQITVNGTVLSSSGTRFAVNFQTGFSGNDIAFHFNPRFEDGGYVVCNTRQNGSWGPEERRTHMPFQKGMPFDLCFLVQSSDFKVMVNGILFVQYFHRVPFHRVDTISVNGSVQLSYISFQNPRTVPVQPAFS

SEQ ID NO: 5 - protein, 148 aa, Homo sapiens
MAFSGSQAPYLSPAVPFSGTIQGGLQDGLQITVNGTVLSSSGTRFAVNFQTGFSGNDIAFHFNPRFEDGGYVVCNTRQNGSWGPEERRTHMPFQKGMPFDLCFLVQSSDFKVMVNGILFVQYFHRVPFHRVDTIFVNGSVQLSYISFQ

SEQ ID NO: 6 - protein, 148 aa, Homo sapiens
MAFSSSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQ

SEQ ID NO: 7 - protein, 158 aa, Mus musculus
MALFSAQSPYINPIIPFTGPIQGGLQEGLQVTLQGTTKSFAQRFVVNFQNSFNGNDIAFHFNPRFEEGGYVVCNTKQNGQWGPEERKMQMPFQKGMPFELCFLVQRSEFKVMVNKKFFVQYQHRVPYHLVDTIAVSGCLKLSFITFQNSAAPVQHVFS

SEQ ID NO: 8 - protein, 159 aa, Rattus norvegicus
MAFFSTQPPYMNPVIPFTGIIQGGLQNGLQITLQGTVHPFPNRIAVNFQTGFSGNDIAFHFNPRFEEGGYVVCNTKQNGKWGPEERKMQMPFQKGMPFELCFLVQRSEFKVMVNKNFFVQYSHRVPYHLVDTISVSGCLHLSFINFQNSTAAPVQPVFS

SEQ ID NO: 9 - protein, 150 aa, Mus musculus
MAYVPAPGYQPTYNPTLPYKRPIPGGLSVGMSFYIQGTAKENMRRFHVNFAVGQDDGADVAFHFNPRFDGWDKVVFNTKQSGRWGKEEEKSMPFQKGKHFELVFMVMPEHYKVVVNGSPFYEYGHRLPVQMVTHLQVDGDLELQSINFFG

SEQ ID NO: 10 - protein, 151 aa, Homo sapiens
MAYVPAPGYQPTYNPTLPYYQPIPGGLNVGMSVYIQGVASEHMKRFFVNFVVGQDPGSDVAFHFNPRFDGWDKVVFNTLQGGKWGSEERKRSMPFKKGAAFELVFIVLAEHYKVVVNGNPFYEYGHRLPLQMVTHLQVDGDLQLQSINFIG

SEQ ID NO: 11 - protein, 153 aa, Homo sapiens
MLSLNNLQNIIYNPVIPFVGTIPDQLDPGTLIVIRGHVPSDADRFQVDLQNGSSVKPRADVAFHFNPRFKRAGCIVCNTLINEKWGREEITYDTPFKREKSFEIVIMVLKDKFQVAVNGKHTLLYGHRIGPEKIDTLGIYGKVNIHSIGFSFS

SEQ ID NO: 12 - protein, 151 aa, Sus scrofa
MAFVPAPGYQPTYNPTLPYYKPIPGGLRVGMSVYIQGVANEHMKRFFVNFVVGQGPGADVAFHFNPRFDGWDKVVFNSQQDGKWGNEEKKRSMPFRKAPAFELVIMVLPEHYKVVVNGDPFYEFGHRIPVQLVTHLQVDGDLTLQSINFIG

SEQ ID NO: 13 - protein, 135 aa, Homo sapiens
SNVPHKSSLPEGIRPGTVLRIRGLVPPNASRFHVNLLCGEEQGSDAALHFNPRLDTSEVVFNSKEQGSWGREERGPGVPFQRGQPFEVLIIASDDGFKAVVGDAQYHHFRHRLPLARVRLVEVGGDVQLDSVRIF

SEQ ID NO: 14 - protein, 144 aa, Rattus norvegicus
MALQFEAFCAGGLAPGWSLTVQGHADAGEDKFEINFLTDAGDIAFHVKPRFSSATVVGNAFQGGRWGQEEVSSVFPLTLGEPFEVEVSADTEHFHIYAQEQKVLQFPHRHRPLATITRVRVLSDHQLAQVELAKRGLSWGDGGY

SEQ ID NO: 15 - protein, 144 aa, Rattus norvegicus
SSFSTQTPYPNLAVPFFTSIPNGLYPSKSIVISGVVLSDAKRFQINLRCGGDIAFHLNPRFDENAVVRNTQINNSWGPEERSLPGSMPFSRGQRFSVWILCEGHCFKVAVDGQHICEYSHRLMNLPDINTLEVAGDIQLTHVET

SEQ ID NO: 16 - protein, 134 aa, Ovis aries
ACGLVASNLNLKPGECLRVRGEVAADAKSFSLNLGKDDNNLCLHFNPRFNAHGDINTIVCNSKDGGAWGAEQRETAFPFQPGSVAEVCISFNQTDLTIKLPDGYEFKFPNRLNLEAINYLSAGGDFKIKCVAFE

SEQ ID NO: 17 - protein, 134 aa, Bos taurus
ACGLVASNLNLKPGECLRVRGEVAADAKSFLLNLGKDDNNLCLHFNPRFNAHGDVNTIVCNSKDAGAWGAEQRESAFPFQPGSVVEVCISFNQTDLTIKLPDGYEFKFPNRLNLEAINYLSAGGDFKIKCVAFE

SEQ ID NO: 18 - protein, 123 aa, Sus scrofa
  misc_feature (24)..(77): “Xaa” is an any amino acid
MSGKVEIMNMDMKVGKTLKIKGKXDDDADGFXINLGQGTDKLALHFXPRFGESTIVCNXRDGNXWGKEQRDSHMXFXPGSEVKLIVTFEEDGFKVKLPDGHQLTFPNRLGYSHLRYLSVQGGF

SEQ ID NO: 19 - protein, 168 aa, Homo sapiens
GPPSGPGAYPSSGQPSAPGAYPATGPYGAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDLTSASYTMI

SEQ ID NO: 20 - protein, 141 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNVSYLKR

SEQ ID NO: 21 - protein, 137 aa, Ovis aries
MDSLPNPYLQSVSLTVCYMVKIKANLLSPFGKNPELQVDFGTGTGQGGNIPFRFWYCDGMVVMNTFTDGSWQKEEKVLTDAFVPGQPFELQFLVLEKEYQVFVKNKPICQFAHRLPLQSVKMLDVRGDIVLTSVDTL

SEQ ID NO: 22 - DNA, 24 bp, Artificial sequence
  PCR primer
ggtaccgcagtctacgagaccagt

SEQ ID NO: 23 - DNA, 18 bp, Artificial sequence
  PCR primer
atgagtcctgaccgaaag

SEQ ID NO: 24 - DNA, 28 bp, Artificial sequence
  PCR primer
attcctgttgcagaagtctacctggaca

SEQ ID NO: 25 - DNA, 24 bp, Artificial sequence
  PCR primer
gaacatcttccacacggtaggggt

SEQ ID NO: 26 - DNA, 30 bp, Artificial sequence
  PCR primer
aggatccatgcagagcgaaagtggtcacga

SEQ ID NO: 27 - DNA, 30 bp, Artificial sequence
  PCR primer
cggcggccgcttaaatctggaagctgatat

(19) United States
(12) Patent Application Publication
(10) Pub. No.: US 2006/0057665 A1
(43) Pub. Date: Mar. 16, 2006

(54) Galectin-14 therapeutic molecule and uses thereof

(75) Inventors: Andrew Nash, Kew (AU); Jillian Lee Dunnphy, Esmont, VA (US); Garry Barcham, Ashwood (AU); Anna Rachel Young, Brunswick (AU); Elza Nicole Theresia Meeusen, Seddon (AU)

Correspondence Address: SEED INTELLECTUAL PROPERTY LAW GROUP PLLC, 701 FIFTH AVE, SUITE 6300, SEATTLE, WA, 98104-7092, US

(73) Assignee: The University of Melbourne, Parkville (AU)

(21) Appl. No.: 10/484,516

(22) PCT Filed: Jul. 19, 2002
(86) PCT No.: PCT/AU02/00965

§ 371 Date: Dec. 14, 2004

(30) Foreign Application Priority Data
Jul. 19, 2001 (AU) PR 6474

(51) Int. Cl. C12P 21/06; C07H 21/02; C07K 14/72

(52) U.S. Cl. 435/69.1; 536/23.1; 435/320.1; 435/325; 530/395

(57) ABSTRACT

The present invention relates generally to a novel galectin and to derivatives, homologues, analogues, chemical equivalents and mimetics thereof capable of modulating an immune response and, in particularly, an inflammatory response. More particularly, the present invention relates to ecalectin-like galectin (herein referred to as “galectin-14”) and to derivatives, homologues, analogues, chemical equivalents and mimetics of said protein sequence. The present invention also contemplates genetic sequences encoding said galectin and derivatives, homologues, analogues, chemical equivalents and mimetics thereof. The molecules of the present invention are useful in a range of therapeutic, prophylactic and diagnostic applications.

### FIELD OF THE INVENTION

The present invention relates generally to a novel galectin and to derivatives, homologues, analogues, chemical equivalents and mimetics thereof capable of modulating an immune response and, in particularly, an inflammatory response. More particularly, the present invention relates to ecalectin-like galectin (herein referred to as “galectin-14”) and to derivatives, homologues, analogues, chemical equivalents and mimetics of said protein sequence. The present invention also contemplates genetic sequences encoding said galectin and derivatives, homologues, analogues, chemical equivalents and mimetics thereof. The molecules of the present invention are useful in a range of therapeutic, prophylactic and diagnostic applications.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

The immune response of mammals to multicellular parasite infections and allergens often involves recruitment of eosinophils (Balic et al., 2000; Kay, A. B., Barata, L., Meng, Q., Durham, S. R. and Ying, S. (1997) Int. Allergy Immunol. 113:196-199). However, the effects of eosinophilia in TH2 allergic-type immune responses remains controversial. Little is known of the putative role eosinophil constituents play in combating multicellular parasites and exacerbating allergic responses, including the role of major constituents such as galectin-10, also known as the Charcot-Leyden crystal (Ackerman, S. J., Corette, S. E., Rosenberg, H. F., Bennett, J. C., Mastrianni, D. M., Nicholson-Weller, A., Weller, P. F., Chin, D. T., and Tennen, D. G. (1993) J. Immunol. 150:456-468; Giembycz and Lindsay, 1999).

Accordingly, there is an ongoing need to identify, and elucidate the functional activity of novel eosinophil-related molecules in order to facilitate the progression towards the more sensitive control of immune responses, such as those involving an inflammatory response.

Sheep have been used successfully in many immunological studies to help define the immune response of large animals, including humans. The advantages of using this animal model are the comparable size and close physiological and phylogenetic relationship with humans compared to rodent models. Haenzonchus contortus is a natural nematode parasite of sheep, which inhabits the abomasum (true stomach). H. contortus challenge infection of immunised sheep can result in massive infiltration of eosinophils within abomasal tissue (Balic et al., 2000 supra), indicating that this tissue may be a good source of inflammatory molecules associated with eosinophil recruitment. The isolation of the inflammatory cells from the abomasal tissue is however problematic and requires tissue digestion. Previous studies have shown that infusion of H. contortus larvae or lipopolysaccharide (LPS) into the mammary gland provides a ready supply of eosinophils and neutrophils, respectively after subsequent “milking” of the gland, without invasive techniques and little discomfort to the animal (Greenhalgh, C. J., Jacobs, H. J. and Meeusen, E. N. T. (1996) Immun. Cell Biol. 74:497-503, Rainbird, M. A., Macmillan, D., and Meeusen, E. N. (1998) Parasite Immunol. 20(2):93-103). This model allows the study of leukocyte populations before and after challenge with allergens, with minimal cell manipulation (Adams and Colditz, 1991, Greenhalgh et al., 1996 supra).

In work leading up to the present invention the inventors have identified a novel galectin that is expressed by circulating eosinophils, or eosinophils migrating into the mammary lavage (MAL), bronchoalveolar lavage (BAL), lung or gastro-intestinal tissue in response to helminth infection or allergen challenge.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The subject specification contains nucleotide and amino acid sequence information prepared using the programme PatentIn Version 3.1, presented herein after the bibliography. Each nucleotide or amino acid sequence is identified in the sequence listing by the numeric indicator <210> followed by the sequence identifier (e.g. <210>1, <210>2, etc). The length, type of sequence (DNA, protein (PRT), etc) and source organism for each nucleotide or amino sequence are indicated by information provided in the numeric indicator fields <211), <212> and <213>, respectively. Nucleotide and amino acid sequences referred to in the specification are defined by the information provided in numeric indicator field <400> followed by the sequence identifier (e.g. <400>1, <400>2, etc).

One aspect of the present invention provides an isolated nucleic acid molecule or derivative, homologue or analogue thereof comprising a nucleotide sequence encoding a novel galectin protein or a derivative, homologue or mimetic thereof wherein said galectin comprises one carbohydrate recognition domain.

Another aspect of the present invention provides a nucleic acid molecule or derivative, homologue or analogue thereof comprising a nucleotide sequence encoding, or a nucleotide sequence complementary to a nucleotide sequence encoding, an amino acid sequence substantially as set forth in <400>2 or a derivative, homologue or mimetic thereof or having at least about 45% or greater similarity to at least 10 contiguous amino acids in <400>2.

In another aspect the present invention contemplates a nucleic acid molecule or derivative, homologue or analogue thereof comprising a nucleotide sequence substantially as set forth in <400>1 or a derivative, homologue or analogue thereof, or capable of hybridising to <400>1 under low stringency conditions.

A further aspect of the present invention contemplates a nucleic acid molecule or derivative, homologue or analogue thereof comprising a nucleotide sequence substantially as set forth in <400>1 or a derivative thereof or capable of hybridising to <400>1 under low stringency conditions and which encodes an amino acid sequence corresponding to an amino acid sequence set forth in <400>2 or a sequence having at least about 45% similarity to at least 10 contiguous amino acids in <400>2.

Yet another aspect of the present invention contemplates a nucleic acid molecule comprising a sequence of nucleotides substantially as set forth in <400>1.

Still another aspect of the present invention is directed to an isolated protein selected from the list consisting of:
(i) A novel galectin protein or a derivative, homologue, analogue, chemical equivalent or mimetic thereof wherein said galectin comprises one carbohydrate recognition domain.
(ii) A protein having an amino acid sequence substantially as set forth in <400>2 or a derivative, homologue or mimetic thereof or a sequence having at least about 45% similarity to at least 10 contiguous amino acids in <400>2 or a derivative, homologue, analogue, chemical equivalent or mimetic of said protein.
(iii) A protein encoded by a nucleotide sequence substantially as set forth in <400>1 or a derivative, homologue or analogue thereof or a sequence encoding an amino acid sequence having at least about 45% similarity to at least 10 contiguous amino acids in <400>2 or a derivative, homologue, analogue, chemical equivalent or mimetic of said protein.
(iv) A protein encoded by a nucleic acid molecule capable of hybridising to the nucleotide sequence as set forth in <400>1 or a derivative, homologue or analogue thereof under low stringency conditions and which encodes an amino acid sequence substantially as set forth in <400>2 or a derivative, homologue or mimetic thereof or an amino acid sequence having at least about 45% similarity to at least 10 contiguous amino acids in <400>2.
(v) A protein as defined in paragraphs (i) or (ii) or (iii) or (iv) in a homodimeric form.
(vi) A protein as defined in paragraphs (i) or (ii) or (iii) or (iv) in a heterodimeric form.

Still yet another aspect of the present invention provides a method for modulating expression of galectin-14 in a subject, said method comprising contacting the galectin-14 gene with an effective amount of an agent for a time and under conditions sufficient to up-regulate or down-regulate or otherwise modulate expression of galectin-14.

Another aspect of the present invention contemplates a method of modulating activity of galectin-14 in a mammal, said method comprising administering to said mammal a modulating effective amount of an agent for a time and under conditions sufficient to increase or decrease galectin-14 functional activity.

In another aspect there is provided a method of treating a mammal said method comprising administering to said mammal an effective amount of an agent for a time and under conditions sufficient to modulate the expression of galectin-14 or sufficient to modulate the activity of galectin-14 wherein said modulation results in modulation of immune functioning.

In yet another aspect the present invention relates to a method of treating a mammal said method comprising administering to said mammal an effective amount of galectin-14 or galectin-14 for a time and under conditions sufficient to modulate immune functioning.

A further aspect of the present invention relates to a method for treatment and/or prophylaxis of a condition characterised by an aberrant, unwanted or otherwise inappropriate inflammatory response in a mammal said method comprising administering to said mammal an effective amount of an agent for a time and under conditions sufficient to modulate the expression of galectin-14 or sufficient to modulate the activity of galectin-14 wherein said modulation results in modulation of said inflammatory response.

In another aspect the present invention relates to a method for the treatment and/or prophylaxis of a condition characterised by an aberrant, unwanted or otherwise inappropriate inflammatory response in a mammal said method comprising administering to said mammal an effective amount of galectin-14 or galectin-14 for a time and under conditions sufficient to modulate said inflammatory response.

In yet another aspect, the present invention provides a method for the treatment and/or prophylaxis of an allergic condition, said method comprising administering to said mammal an effective amount of an agent for a time and under conditions sufficient to modulate the expression of galectin-14 or sufficient to modulate the activity of galectin-14 for a time and under conditions sufficient to down-regulate a Th-2 type inflammatory response.

In still yet another aspect there is provided a method for the treatment and/or prophylaxis of an allergic condition, said method comprising administering to said mammal an effective amount of galectin-14 or galectin-14 for a time and under conditions sufficient to up-regulate a Th-2 type inflammatory response.

Yet another aspect of the present invention relates to the use of an agent capable of modulating the expression of galectin-14 or modulating the activity of galectin-14 in the manufacture of a medicament for the modulation of an inflammatory response.

A further aspect of the present invention relates to the use of galectin-14 or galectin-14 in the manufacture of a medicament for the modulation of an inflammatory response.

Still yet another aspect of the present invention relates to agents for use in modulating galectin-14 expression or galectin-14 activity wherein said modulation results in modulation of an inflammatory response.

Another aspect of the present invention relates to galectin-14 or galectin-14 for use in modulating an inflammatory response.

In yet another further aspect the present invention contemplates a pharmaceutical composition comprising galectin-14, galectin-14 or an agent capable of modulating galectin-14 expression or galectin-14 activity together with one or more pharmaceutically acceptable carriers and/or diluents. Galectin-14, galectin-14 or said agent are referred to as the active ingredients.

Another aspect of the present invention provides a method for detecting an agent capable of modulating the function of galectin-14 or its functional equivalent or derivative thereof said method comprising contacting a cell or extract thereof containing said galectin-14 or its functional equivalent or derivative with a putative agent and detecting an altered expression phenotype associated with said galectin-14 or its functional equivalent or derivative.

In yet another aspect the present invention provides a method for detecting an agent capable of binding or otherwise associating with a galectin-14 binding site or functional equivalent or derivative thereof said method comprising contacting a cell containing said galectin-14 binding site or functional equivalent or derivative thereof with a putative agent and detecting an altered expression phenotype associated with modulation of the function of galectin-14 or its functional equivalent or derivative.

Still another aspect of the present invention is directed to antibodies to galectin-14 including catalytic antibodies.

In another aspect of the present invention, the molecules of the present invention are also useful as screening targets for use in applications such as the diagnosis of disorders which are regulated by galectin-14.

Single and three letter abbreviations used throughout the specification are defined in Table 1.

TABLE 1
Single and three letter amino acid abbreviations

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any residue | Xaa | |

### BRIEF DESCRIPTION OF THE DRAWINGS

FIG. 1 is a schematic representation of the nucleotide <400>1 and predicted amino acid <400>2 sequence of ovine galectin-14. Three putative casein kinase II sites and two putative protein kinase C sites are shown (dark and light shading, respectively). A putative N-glycosylation site is underlined, and a Bcl-2-like motif is double underlined. RT-PCR primers used to amplify the cDNA and confirm the coding region from three individual animals are boxed. Putative binding sites for transcription factors within the untranslated regions are also shown (TCFII and STT5). These putative binding sites were detected using MatInspector V2.2, available at www.gsf.de/cgi-bin/matsearch.pl. Please note, the untranslated regions have not been confirmed in multiple animals, but have been confirmed in multiple cDNA from one cDNA library. The original partial clone isolated by differential display was flanked by TaqI restriction enzyme sites (shown in bold-face). This partial clone was used as the probe in all Northern blots.

FIG. 2 is a schematic representation of the comparison of the amino acid sequences of known galectins to the predicted amino acid sequence of galectin-14. Amino acids identical to the corresponding amino acid in galectin-14 are highlighted. The asterisks (*) present identical amino acid in all proteins. The dots (.) represent conserved amino acids in all proteins. The arrowed residues are thought to be important for binding of carbohydrates by galectins (Hirabayashi, J. and Kasai, K. (1994) Glycoconj. J. 11(5):437-442; Ahmed, H., Fink, N. E., Pohl, J., and Vasta, G. R. (1996) Biochem. Biophys. Res. Commun. 68:650-657). The first 79 residues of human galectin-3 are not shown. The tandem-repeat type galectins, such as the galectin-9 sequences, continue for another 175-206 residues, which includes their second CRD. GI, gastrointestinal isoform.

FIG. 3 is an image of Northern blot analysis of galectin-14 mRNA levels. Northern blot hybridisation of the partial galectin-14 clone to total RNA prepared from MAL cells that mad migrated into the mammary gland in response to H. contortus intramammary infusion. Cells were uncultured (0), or cultured in the presence of recombinant IL-5 for five (5), or 24 h (24).

FIG. 4 is an image of Northern blot analysis of galectin-14 mRNA levels. Northern blot hybridisation of the partial galectin-14 clone to total RNA prepared from eosinophil-rich MAL cells that had migrated into the mammary gland in response to LPS or HDME intramammary infusions. Cells were collected from the LPS treated glands at 24 h or 5 days post-intramammary infusion (24 h or 5 d respectively), and consisted predominantly of neutrophils (24 h) or macrophages (5 d). Cells collected from the HDME treated glands 48 h post-intramammary infusion (48 h) were predominantly eosinophils.

FIG. 5 is an image of Northern blot analysis of galectin-14 mRNA levels. Northern blot hybridisation of the partial galectin-14 clone to total RNA prepared from lung tissue (L) or BAL cells (B) of untreated sheep (control), or sheep that had received HDME challenge in the left lung lobe (Sheep 1-3). In the HDME challenged sheep the left and right lung tissue and BAL was collected separately (left and right respectively). In the left BAL, eosinophils represented 5, 38 and 10% of total cells in Sheep 1, 2 and 3 respectively.

FIG. 6 is an image of immunohistology of tissue sections or cytospots. Tissue sections of H. contortus infected abomasum, or lung sections from HDME treated sheep were stained with anti-galectin-14 polyclonal serum. Cytospots of H. contortus intramammary infusion MAL leukocytes were stained with anti-galectin-14 monoclonal supernatant and eosin Y, or eosin Y alone as a negative control. In both tissue sections and cytospots galectin-14 appeared to localise to eosinophils.

FIG. 7 is an image of Western blot analysis of eosinophil proteins. An eosinophil-rich MAL cell population was extracted from a mammary gland 48 h post-H. contortus intramammary infusion. The MAL cells were solubilized in reducing sample buffer, run on 12.5% SDS-PAGE, transferred to a nitrocellulose filter and then stained with polyclonal anti-galectin-14 serum (P), eight separate galectin-14 mAbs (1-8), or anti-OVGAL11 mAb as a negative control (coneg). The polyclonal serum and seven of the eight anti-galectin-14 mAbs react with native galectin-14.

FIG. 8 is an image of Western blot analysis of MAL fluid or stomach mucus scrapings associated with eosinophilia. MAL fluid supernatant (without cells) was obtained from sheep that had received H. contortus intramammary infusions, and stomach mucus scrapings were collected from immunised sheep 10 days post H. contortus challenge infection. The membrane was stained with anti-galectin-14 polyclonal serum. Recombinant galectin-14 (rgalectin-14) was included as a positive control. A similar result was seen using anti-galectin-14 mAbs.

FIG. 9 is an image of the Northern blot analysis of galectin-14 mRNA levels in isolated leukocytes and whole tissue. Total RNA from macrophage-(M), neutrophil-(N), or eosinophil-(E) rich MAL cell populations, or from lung tissue (L), or BAL cells (B) were used. The lung tissue and BAL cells were collected from sheep that had been sensitized with HDM and challenged 48 hrs earlier in the left lung lobe with HDM, and in the right lung lobe with sterile PFS (Treated sheep). Control sheep received sterile PFS only in both lung lobes (Controls). 18S rRNA is shown to correct for loading errors. Results shown are representative of 3 treated and 3 control sheep.

FIG. 10 is an image of SDS-PAGE and Western blot analysis of recombinant galectin-14 and endogenous proteins. Cleaved and purified recombinant galectin-14 (rGal-140 was analyzed by Coomassie Blue-stained SDS-PAGE (A), Western blot using galectin-14 mAb (B and C). Initially in recombinant protein preparations galectin-14 is predominantly a monomer (A and B), but after storage at high concentrations recombinant galectin-14 often self-aggregates into oligomers (C). Western blot analysis using galectin-14 mAb was also performed on endogenous proteins prepared from cell suspenses (˜2×10⁴ cells/lane) containing high proportions (>80%) of MAL eosinophils (E), MAL neutrophils (N), lymph node (LN), lymphocytes (L), or BAL macrophages (M) from control lungs, compared with BAL cells containing 5% eosinophils after local HDM challenge (HDM). The far right panel shows the presence of galectin-14 in cell-free MAL fluid of a sensitized sheep before (S) and after (SC) HDM challenge of the mammary gland. The arrow points to the position of monomeric galectin-14. All samples were run under reducing conditions.

FIG. 11 is an image of the immunocytochemistry of cytospots and tissue sections with galectin-14 mAb. Immunostaining (brown) of cytospots prepared from HDM challenged BAL (A) and MAL (B) cells, or tissue sections of HDM challenged left lung (C) and saline challenged right lung (D). The cytospots and tissue sections were counter stained with eosin Y (pink) to identify eosinophils. Magnification: A and B ×200; C and D ×40.

FIG. 12 is a graphical representation of the flow cytometry analysis of leukocytes after intracellular staining with galectin-14 mAb. MAL eosinophils (A), peripheral blood neutrophils (B), MAL lymphocytes (C), or MAL macrophages (D) were gated on forward and side scatter profile. The solid histogram in each plot represents staining with galectin-14 mAb. Binding of isotype-matched control mAb is shown by the open histograms. Profiles shown are representative of three separate experiments.

FIG. 13 is an image of the Western blot analysis of cell-free BAL fluid using galectin-14 mAb. BAL fluid was collected before (0 h), or 6, 24, or 48 hours post-local lung challenge of HDM sensitized sheep. The left lung lobe was challenged with HDM, and the right lung lobe with sterile PFS as a control. The total number of cells in the BAL fluid and the number of eosinophils present at each time point was calculated (A), and cell-free BAL fluid was probed with galectin-14 mAb (B).

FIG. 14 is an image of a hemagglutination assay using GST-galectin-14 fusion protein. Agglutination of rabbit erythrocytes was assayed in a 96-well microtiter plate. Assays were conducted in the presence (2, 3, 4, and 5) or absence (1 and 6) of 0.28 μM recombinant GST-galectin-14, the minimum concentration to induce agglutination. Increasing concentrations (0.78-100 mM) of lactose (2), galactose (3), or N-acetyl-glucosamine (4) were added to inhibit agglutination. In the presence of GST-galectin-14 without sugar (5) the rabbit erythrocytes form a “mat” at the bottom of the wells. The assay buffer DES alone (1) and 3 μM rGST (6) were included as negative controls.

FIG. 15 is an image of antibody inhibition of GST-galectin-14 fusion protein hemagglutination activity. Agglutination of rabbit erythrocytes was assayed in a 96-well microtiter plate. Assays were conducted in the presence of 0.059 μM recombinant GST-galectin-14, the minimum concentration to induce agglutination, and increasing concentrations of antibodies raised to recombinant galectin-14 were added to inhibit agglutination. The antibodies added were doubling dilutions of ascitic fluid ( 1/25,600 to 1/200) from monoclonal antibody clones 1.3 (3), 1.2 (4), 3.2 (5), 3.6 (6), polyclonal rabbit sera ( 1/512 to ¼) (7), and supernatants ( 1/512 to ¼) from monoclonal antibody clones 1.4 (8), 1.9 (9), 3.5 (10) and 3.7 (11). In the presence of GST-galectin-14 without antibody (2) the rabbit erythrocytes form a “mat” at the bottom of the wells. The assay buffer DES alone (1) was included as a negative control.

FIG. 16 is an image of antibody potentiation of GST-galectin-14 fusion protein hemagglutination activity. Agglutination of rabbit erythrocytes was assayed in a 96-well microtiter plate. Assays were conducted in the presence of 0.042 μM recombinant GST-galectin-14, the maximum concentration which did not induce agglutination, and increasing concentrations of antibodies raised to recombinant galectin-14 were added to potentiate agglutination. The antibodies added were doubling dilutions of ascitic fluid ( 1/51,200 to 1/400) from monoclonal antibody clones 1.3 (3), 1.2 (4), 3.2 (5), 3.6 (6), polyclonal rabbit sera ( 1/512 to ¼) (7), and supernatants ( 1/512 to ¼) from monoclonal antibody clones 1.4 (8), 1.9 (9), 3.5 (10) and 3.7 (11). In the presence of GST-galectin-14 without antibody (2) the rabbit erythrocytes fail to form a “mat” at the bottom of the wells. The assay buffer DES alone (1) was included as a negative control.

FIG. 17 is an image of the saturation binding of GST-galectin-14 fusion protein to immobilised laminin. Laminin binding of GST-galectin-14 fusion protein was assayed using a solid-phase binding assay in a 96-well microtitre plate. Serial dilutions of GST-galectin-14 (▪) and GST (▴) were added to microtitre wells containing laminin. 0.3 M Lactose was added to GST-galectin-14 to inhibit binding (◯). The level of bound protein was monitored by the binding of rabbit anti-GST HRP antibody developed with TMB substrate and detected by a change in absorbance at 450 nm.

### DETAILED DESCRIPTION OF THE INVENTION

The present invention is predicated, in part, on the identification and cloning of a novel galectin molecule termed ecalectin-like galectin (herein referred to as “galectin-14”). The isolation of this molecule permits the identification and design of a range of products for use in therapy, diagnosis and for antibody generation. These therapeutic molecules may also act as either antagonists or agonists of galectin function and would be useful inter alia, for the modulation of the immune response and, in particular, for the modulation of inflammation in conditions characterised by an unwanted or inappropriate inflammatory response, such as occurs during an allergic reaction.

Accordingly, one aspect of the present invention provides an isolated nucleic acid molecule or derivative, homologue or analogue thereof comprising a nucleotide sequence encoding a novel galectin protein or a derivative, homologue or mimetic thereof wherein said galectin comprises one carbohydrate recognition domain.

Reference to a “carbohydrate recognition domain” should be understood as a reference to a protein domain which comprises conserved amino acid residues with sugar binding motifs. The carbohydrate recognition domain may be continuous, meaning that it is comprised of a continuous sequence of amino acids, or it may be discontinuous, meaning that it is comprised of individual amino acids or sequences of amino acids from two or more separate regions of the protein and which are brought into proximity of one another, to form the carbohydrate recognition domain, due to the secondary, tertiary or quaternary structure of the protein.

Reference to a “galectin” should be understood as a reference to a molecule of the family of β-galactoside binding proteins that exhibit, inter alia, growth regulatory and immunomodulatory properties (Vasta, G. R., Quenenberry, M., Ahmed, H. and O'Leary, N. (1999) Dev. Comp. Immunol. 23:401-420).

More particularly, the present invention provides a nucleic acid molecule or derivative, homologue or analogue thereof comprising a nucleotide sequence encoding, or a nucleotide sequence complementary to a nucleotide sequence encoding, an amino acid sequence substantially as set forth in <400>2 or a derivative, homologue or mimetic thereof or having at least about 45% or greater similarity to at least 10 contiguous amino acids in <400>2.

The term “similarity” as used herein includes exact identity between compared sequences at the nucleotide or amino acid levels. Where there is non-identity at the nucleotide level “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particular preferred embodiment, nucleotide and sequence comparisons are made at the level of identity rather than similarity. Any number of programs are available to compare nucleotide and amino acid sequences. Preferred programs have regard to an appropriate alignment. One such program is Gap which considers all possible alignment and gap positions and creates an alignment with the largest number of matched bases and the fewest gaps. Gap uses the alignment method of Needleman and Wunsch. Gap reads a scoring matrix that contains values for every possible GCG symbol match. GAP is available on ANGIS (Australian National Genomic Information Service) at website http://mel1.angis.orz.au.

In another embodiment the present invention contemplates a nucleic acid molecule or derivative, homologue or analogue thereof comprising a nucleotide sequence substantially as set forth in <400>1 or a derivative, homologue or analogue thereof, or capable of hybridising to <400>1 under low stringency conditions.

Reference herein to a low stringency includes and encompasses from at least about 0% v/v to at least about 15% v/v formamide and from at least about 1M to at least about 2M salt for hybridisation, and at least about 1M to at least about 2M salt for washing conditions.

Alternative stringency conditions may be applied where necessary, such as medium stringency, which includes and encompasses from at least about 16% v/v to at least about 30% v/v formamide and from at least about 0.5M to at least about 0.9M salt for hybridisation, and at least about 0.5M to at least about 0.9M salt for washing conditions, or high stringency, which includes and encompasses from at least about 31% v/v to at least about 50% v/v formamide and from at least about 0.01M to at least about 0.15M salt for hybridisation, and at least about 0.01M to at least about 0.15M salt for washing conditions. Stringency may be measured using a range of temperature such as from about 40° C. to about 65° C. Particularly useful stringency conditions are at 42° C. In general, washing is carried out at Tₘ=69.3+0.41 (G+C) %=−12° C. However, the Tₘ of a duplex DNA decreases by 1° C. with every increase of 1% in the number of mismatched based pairs (Bonner et al (1973) J. Mol. Biol., 81:123).

Preferably, the present invention contemplates a nucleic acid molecule or derivative, homologue or analogue thereof comprising a nucleotide sequence substantially as set forth in <400>1 or a derivative thereof or capable of hybridising to <400>1 under low stringency conditions and which encodes an amino acid sequence corresponding to an amino acid sequence set forth in <400>2 or a sequence having at least about 45% similarity to at least 10 contiguous amino acids in <400>2.

More particularly, the present invention contemplates a nucleic acid molecule comprising a sequence of nucleotides substantially as set forth in <400>1.

The nucleic acid molecule according to this aspect of the present invention corresponds herein to “galectin-14”. This gene has been determined, in accordance with the present invention, to encode a protein belonging to the galectin family of molecules. The product of the galectin-14 gene is referred to herein as galectin-14. Without limiting the present invention to any one theory or mode of action the subject galectin-14 protein demonstrates similarity to the tandem-repeat type galectin galectin-9/ecalectin (Hirashima, M. (2000) Int. Arch. Allergy Immunol. 122 (Suppl 1):6-9) and hence was named ecalectin-like galectin (galectin-14). However galectin-14 is not thought to represent the ovine homologue of galectin-9/ecalectin since the highest overall amino acid identity it exhibits to any galectin-9/ecalectin sequence is only 58%. Across species the galectins are well conserved, for example human and sheep galectin-1 share 87% amino acid identity. Additionally, within the same region compared to galectin-14, the galectin-9 variants of human and rat share 67%, and human and mouse 68.5% amino acid identity. A comparison between galectin-14 and galectin-9 variants also reveals that there are 23 non-conservative substitutions of residues that are conserved in the three galectin-9 sequences cloned from different species (human, mouse and rat; Türeci, O., Schmitt, H., Fadle, N., Pfreundschuh, M and Sahin, U (1997) J. Biol. Chem. 272:6416-6422; Matsumoto, R., Matsumoto, H., Seki, M., Hata, M., Asano, Y., Kanegasaki, S., Stevens, R. L. and Hirashima, M. (1998) J. Biol. Chem. 273:16976-16984; Wada, J. and Kanwar, Y. S. (1997) J. Biol. Chem. 272:6078-6086; Leal-Pinto, E., Wenjing, T., Rappaport, J. Richardson, M., Knorr, B. A. and Abramson, R. G. (1997) J. Biol. Chem. 272:617-625). Unlike the galectin-9 variants, which are tandem-repeat type galectins, galectin-14 is clearly a proto-type galectin containing only one carbohydrate recognition domain. In addition to being truncated after only one carbohydrate recognition domain, galectin-14 has an extended NH-terminus (12 residues longer than that of the galectin-9 variants). This extended NH-terminus is unusual for galectins, currently the only other galectin reported to have an extended NH-terminus is galectin-3. However the NH-terminus of galectin-14 is still much shorter than that of galectin-3, and does not contain a proline and glycine rich repetitive sequence, and hence galectin-14 should not be classified as a chimera type galectin.

Still without limiting the present invention in any way, the distribution of galectin-14 mRNA and protein indicates that galectin-14 is not the functional homologue of any of the galectin-9 variants, in that galectin-14 appears to be expressed by eosinophils, not lymphocytes. The only other galectin known to be expressed at high levels in eosinophils is galectin-10, also known as the Charcot-Leyden crystal (Ackerman et al., 1993 supra). However, galectin-14 exhibits little identity to this galectin (25% amino acid identity).

Galectin-14 is thought to bind β-galactosidases and exhibits a similar carbohydrate binding specificity to the most well characterised galectins. It contains six of the seven residues reported to be important for sugar recognition and binding by galectins (Hirabayashi, J. and Kasai, K. (1994) supra; Ahmed, H. et al (1996) supra), and the amino acid substitution that does occur is a conservative change. Additionally, the arginine residues known to be important for sugar binding, hemagglutination and eosinophil chemotactic activity of ecalectin/galectin-9 are also conserved in galectin-14 (R⁶⁵ and R²³⁹ of ecalectin; Matsushita, N., Nishi, N., Seki, M., Matsumoto, R., Kuwabara, I., Liu, F.-T., Hata, Y., Nakamura, T. and Hirashima, M. (2000) J. Biol. Chem. 275:8355-8360). The sugar binding activity of ecalectin appears to be involved in the eosinophil chemotactic activity, however both carbohydrate recognition domains are required (Matsushita et al., 2000 supra). Hence it is thought that galectin-14 is not likely to exhibit chemotactic activity via a similar mechanism to ecalectin. It is thought that galectin-14 regulates chemotaxis however, perhaps even acting as an antagonist to ecalectin.

The galectin-14 sequence contains a Bcl-2 like motif shared with galectins 1 and 3. Recently it has been suggested that galectins 1 and 3 may regulate apoptosis through this motif (Yang, R. Y., Hsu, D. K. and Liu, F. T. (1996) Proc. Natl. Acad. Sci. USA 93:6737-6742). Galectin-3 contains a perfect NWGR motif which is conserved in the Bcl-2 family. It has been postulated that Bcl-2 and galectin-3 may heterodimerise to inhibit Fas-antibody mediated apoptosis (Yang et al., 1996 supra;, Akahani, S., Nangia-Makker, P., Inohara, H., Kim, H. R. and Raz, A. (1997) Cancer Res. 57:4272-5276; Perillo, N. L., Uittenbogaart, C. H., Nguyen, J. T. and Baum, L. G. (1997) J. Exp. Med. 185:1851-1858). The other galectins have similar but not identical sequences. If this motif is providing the means for regulation of apoptosis by galectins 1 and 3 then galectin-14 is most likely to also exhibit this activity.

Ovine galectin-14 is defined by the amino acid sequence set forth in <400>2. The cDNA nucleotide sequence for ovine galectin-14 is defined by the nucleotide sequence set forth in <400>1. The nucleic acid molecule encoding galectin-14 is preferably a sequence of deoxyribonucleic acids such as a cDNA sequence or a genomic sequence. A genomic sequence may also comprise exons or introns. A genomic sequence may also include a promoter region or other regulatory regions.

Reference herein to galectin-14 and galectin-14 should be understood as a reference to all forms of galectin-14 and galectin-14, respectfully, including, for example, any peptide and cDNA isoforms which arise from alternative splicing of galectin-14 mRNA, mutants or polymorphic variants of galectin-14 or galectin-14, any postranslation modified forms of galectin-14 or any non-postranslational modified forms of galectin-14. To the extent that it is not specified, reference herein to galectin-14 and galectin-14 includes reference to derivatives, homologues, analogues, chemical equivalents and mimetics thereof.

The protein and/or gene is preferably from a human, primate, livestock animal (e.g. sheep, pig, cow, horse, donkey), laboratory test animal (e.g. mouse, rabbit, rat, guinea pig), companion animal (e.g. dog, cat), captive wild animal (e.g. fox, kangaroo, deer), aves (e.g. chicken, geese, duck, emu, ostrich), reptile or fish.

Derivatives include fragments, parts, portions, mutants, variants and mimetics from natural, synthetic or recombinant sources including fusion proteins. Parts or fragments include, for example, active regions of galectin-14. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences including fusions with other peptides, polypeptides or proteins.

Chemical and functional equivalents of galectin-14 or galectin-14 should be understood as molecules exhibiting any one or more of the functional activities of galectin-14 or galectin-14 and may be derived from any source such as being chemically synthesized or identified via screening processes such as natural product screening.

The derivatives of galectin-14 include fragments having particular epitopes or parts of the entire galectin-14 protein fused to peptides, polypeptides or other proteinaceous or non-proteinaceous molecules.

Analogues of galectin-14 contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences may similarly be derived from single or multiple nucleotide substitutions, deletions and/or additions including fusion with other nucleic acid molecules. The derivatives of the nucleic acid molecules of the present invention include oligonucleotides, PCR primers, antisense molecules, molecules suitable for use in cosuppression and fusion of nucleic acid molecules. Derivatives of nucleic acid sequences also include degenerate variants.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carba-moylation of amino groups with cyanate; trinitrobenzylation of amino groups with 2,4,6-trinitrobenzene sulphonic acid (NBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, omithine, sarcosine, 4-amino-3-hydroxy-6-methylheptanoic acid, 2-thienyl alanine and/or D-isomers of amino acids. A list of unnatural amino acid contemplated herein is shown in Table 2.

TABLE 2

| Non-conventional | |
|---|---|
| amino acid | Code |
| α-aminobutyric acid | Abu |
| α-amino-α-methylbutyrate | Mgabu |
| aminocyclopropane- | Cpro |
| carboxylate | |
| aminoisobutyric acid | Aib |
| aminonorbornyl- | Norb |
| carboxylate | |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl) | Nnbhm |
| carbamylmethyl)glycine | |
| 1-carboxy-1-(2,2-diphenyl-Nmbc | |
| ethylamino)cyclopropane | |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl--aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl)glycine | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

The nucleic acid molecule of the present invention is preferably in isolated form or ligated to a vector, such as an expression vector. By “isolated” is meant a nucleic acid molecule having undergone at least one purification step and this is conveniently defined, for example, by a composition-comprising at least about 10% subject nucleic acid molecule, preferably at least about 20%, more preferably at least about 30%, still more preferably at least about 40-50%, even still more preferably at least about 60-70%, yet even still more preferably 80-90% or greater of subject nucleic acid molecule relative to other components as determined by molecular weight, encoding activity, nucleotide sequence, base composition or other convenient means. The nucleic acid molecule of the present invention may also be considered, in a preferred embodiment, to be biologically pure.

The term “protein” should be understood to encompass peptides, polypeptides and proteins. The protein may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “protein” includes a protein comprising a sequence of amino acids as well as a protein associated with other molecules such as amino acids, lipids, carbohydrates or other peptides, polypeptides or proteins.

In a particularly preferred embodiment, the nucleotide sequence corresponding to galectin-14 is a cDNA sequence comprising a sequence of nucleotides as set forth in <400>1 or a derivative or analogue thereof including a nucleotide sequence having similarity to <400>1.

A derivative of a nucleic acid molecule of the present invention also includes a nucleic acid molecule capable of hybridising to a nucleotide sequence as set forth in <400>1 under low stringency conditions. Preferably, low stringency is at 42° C.

The nucleic acid molecule may be ligated to an expression vector capable of expression in a prokaryotic cell (e.g. E. coli) or a eukaryotic cell (e.g. yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector. The latter embodiment facilitates production of recombinant forms of galectin-14 which forms are encompassed by the present invention.

The present invention extends to the expression product of the nucleic acid molecules as hereinbefore defined.

The expression product is a novel galectin molecule having an amino acid sequence set forth in <400>2 or is a derivative, homologue, analogue, chemical equivalent or mimetic thereof as defined above or is a molecule having an amino acid sequence of at least about 45% similarity to at least 10 contiguous amino acids in the amino acid sequence as set forth in <400>2 or a derivative, homologue, analogue, chemical equivalent or mimetic thereof.

Another aspect of the present invention is directed to an isolated protein selected from the list consisting of:
(i) A novel galectin protein or a derivative, homologue, analogue, chemical equivalent or mimetic thereof wherein said galectin comprises one carbohydrate recognition domain.
(ii) A protein having an amino acid sequence substantially as set forth in <400>2 or a derivative, homologue or mimetic thereof or a sequence having at least about 45% similarity to at least 10 contiguous amino acids in <400>2 or a derivative, homologue, analogue, chemical equivalent or mimetic of said protein.
(iii) A protein encoded by a nucleotide sequence substantially as set forth in <400>1 or a derivative, homologue or analogue thereof or a sequence encoding an amino acid sequence having at least about 45% similarity to at least 10 contiguous amino acids in <400>2 or a derivative, homologue, analogue, chemical equivalent or mimetic of said protein.
(iv) A protein encoded by a nucleic acid molecule capable of hybridising to the nucleotide sequence as set forth in <400>1 or a derivative, homologue or analogue thereof under low stringency conditions and which encodes an amino acid sequence substantially as set forth in <400>2 or a derivative, homologue or mimetic thereof or an amino acid sequence having at least about 45% similarity to at least 10 contiguous amino acids in <400>2.
(v) A protein as defined in paragraphs (i) or (ii) or (iii) or (iv) in a homodimeric form.
(vi) A protein as defined in paragraphs (i) or (ii) or (iii) or (iv) in a heterodimeric form.

The protein of the present invention is preferably in isolated form. By “isolated” is meant a protein having undergone at least one purification step and this is conveniently defined, for example, by a composition comprising at least about 10% subject protein, preferably at least about 20%, more preferably at least about 30%, still more preferably at least about 40-50%, even still more preferably at least about 60-70%, yet even still more preferably 80-90% or greater of subject protein relative to other components as determined by molecular weight, amino acid sequence or other convenient means. The protein of the present invention may also be considered, in a preferred embodiment, to be biologically pure.

The galectin-14 of the present invention may be in multimeric form meaning that two or more molecules are associated together. Where the same galectin-14 molecules are associated together, the complex is a homomultimer. An example of a homomultimer is a homodimer. Where at least one galectin-14 is associated with at least one non-galectin-14 molecule, then the complex is a heteromultimer such as a heterodimer.

The ability to produce recombinant galectin-14 permits the large scale production of galectin-14 for commercial use. The galectin-14 may need to be produced as part of a large peptide, polypeptide or protein which may be used as is or may first need to be processed in order to remove the extraneous proteinaceous sequences. Such processing includes digestion with proteases, peptidases and amidases or a range of chemical, electrochemical, sonic or mechanical disruption techniques.

Notwithstanding that the present invention encompasses recombinant proteins, chemical synthetic techniques are also preferred in synthesis of galectin-14.

Galectin-14 according to the present invention is conveniently synthesised based on molecules isolated from a mammal. Isolation of these molecules may be accomplished by any suitable means such as by chromotographic separation, for example using CM-cellulose ion exchange chromotography followed by Sephadex (e.g. G-50 column) filtration. Many other techniques are available including HPLC, PAGE amongst others.

Galectin-14 may be synthesised by solid phase synthesis using F-moc chemistry as described by Carpino et al. (1991). Galectin-14 and fragments thereof may also be synthesised by alternative chemistries including, but not limited to, t-Boc chemistry as described in Stewart et al. (1985) or by classical methods of liquid phase peptide synthesis.

Without limiting the present invention to any one theory or mode of action, galectin-14 is known to accumulate under the plasma membrane. Possibly these patches, which have sometimes been reported in relation to galectins 1 and 3 as evaginations in the plasma membrane, are the localisation of the galectins in caveolae (Cooper, D. N. W. and Barondes, S. H. (1990) J. Cell Biol. 110:1681-1691). Galectin-1and -3 accumulated under the plasma membrane appear to be released into the extracellular space enveloped in vesicles as insoluble particles, and later released from the vesicles as soluble protein by an unknown process (Cooper and Barondes, 1990 supra; Mehul, B. and Hughes, R. C. (1997) J. Cell Sci. 110:1169-1178). Like the other galectins the release of galectin-14 is unlikely to occur via the endoplasmic reticulum-golgi network (Sato et al., 1993). Possibly galectin-14 is also being released into the extracellular environment in vesicles after accumulating under the plasma membrane.

Once in the extracellular environment galectin-14 is thought to bind to extracellular matrix proteins such as laminin. Galectins 1, 3 and 8 are all known to bind to extracellular matrix proteins through their carbohydrate recognition domains (Rabinovich, G. A., Ariel, A., Hershkoviz, R., Hirabayashi, J., Kasai, K.-I. and Lider, O (1999) Immunology 97:100-106; Kuwabara, I. and Liu, F. T. (1996) J. Immunol. 156:3939-3944; Hadari, Y. R., Arbel-Goren, R., Levy, Y., Amsterdam, A., Alon, R., Zakut, R., and Zick, Y. (2000) J. Cell Sci. 113:2385-2397). The interaction between galectins and extracellular matrix components is postulated to regulate cell migration, proliferation and apoptosis (Perillo et al., 1998). The recruitment and activation of eosinophils is also regulated by their interaction with extracellular matrix proteins (Kita et al., 1996).

The localisation of galectin-14 to eosinophils suggests that this molecule plays a role in allergic-type inflammation. Many galectins have already been linked to immunity (Rabinovich, G. A., Daly, G., Dreja, H., Tailor, H., Riera, C. M., Hirabayashi, J. and Chernajovsky, Y. (1999) J. Exp. Med. 190:385-398; Vasta, (1999) supra). They are known to regulate cytokine production (Cortegano, I., del Pozo, V., Carbada, B., de Andres, B., Gallardo, S., del Amo, A., Arrieta, I., Jurado, A., Palomino, P., Liu, F. T. and Lahoz, C. (1998) J. Immunol. 161:385-389; Vespa, g. N., Lewis, L. A., Kozak, K. R., Moran, M., Nguyen, J. R., Baum, L. G. and Miceli, M. C. (1999) J. Immunol. 162:799-806; Rabinovich, 1999a supra), thymocyte apoptosis (Chung et al., 2000, Pace et al., 2000), activation of neutrophils and mast cells (Yamaoka, A., Kuwabara, I., Frigeri, L. G. and Liu, F. T. (1995) J. Immunol. 154:3479-3487; Liu, 1993), and migration of leukocytes (Sano, H., Hsu, D. K., Yu, L., Apgar, J. R., Kuwabara, I., Yamanaka, T., Hirashima, M. and Liu, F. T. (2000) J. Immunol. 165:2156-2164; Matsushita et al., 2000 supra). Galectin-14 is thought to effect TH₂-type inflammation. The presence of galectin-14 in eosinophils and it's increased mRNA expression during inflammatory responses indicates that this protein plays an important role in allergic-type immune responses in mammals.

Accordingly, the cloning and sequencing of this gene and its expression product now provides an additional gene for use in modulating immune response and in the therapeutic and prophylactic treatment of conditions characterised by an aberrant, unwanted or otherwise inappropriate inflammatory response, such as occurs in allergic reactions. Accordingly, the present invention contemplates therapeutic and prophylactic uses of galectin-14 amino acid and nucleic acid molecules, in addition to galectin-14 agonistic and antagonistic agents, for the regulation of immune responses and, in particular, inflammatory responses.

The present invention contemplates, therefore, a method for modulating expression of galectin-14 in a subject, said method comprising contacting the galectin-14 gene with an effective amount of an agent for a time and under conditions sufficient to up-regulate or down-regulate or otherwise modulate expression of galectin-14. For example, galectin-14 antisense sequences such as oligonucleotides may be introduced into an eosinophil to down-regulate the expression of this molecule. Conversely, a nucleic acid molecule encoding galectin-14 or a derivative, homologue or mimetic thereof may be introduced to induce or up-regulate the galectin-14 production of any cell not expressing the endogenous galectin-14 gene.

Another aspect of the present invention contemplates a method of modulating activity of galectin-14 in a mammal, said method comprising administering to said mammal a modulating effective amount of an agent for a time and under conditions sufficient to increase or decrease galectin-14 functional activity.

Modulation of said activity by the administration of an agent to a mammal can be achieved by one of several techniques including, but in no way limited to, introducing into said mammal a proteinaceous or non-proteinaceous molecule which:
(i) modulates expression of galectin-14;
(ii) functions as an antagonist of galectin-14;
(iii) functions as an agonist of galectin-14 (including administration of galectin-14 or functional equivalent, derivative, homologue, analogue or mimetic thereof).

Said proteinaceous molecule may be derived from natural or recombinant sources including fusion proteins or following, for example, natural product screening. Said non-proteinaceous molecule may be, for example, a nucleic acid molecule or may be derived from natural sources, such as for example natural product screening or may be chemically synthesised. The present invention contemplates chemical analogues of galectin-14 or small molecules capable of acting as agonists or antagonists of galectin-14. Chemical agonists may not necessarily be derived from galectin-14 but may share certain conformational similarities. Alternatively, chemical agonists may be specifically designed to mimic certain physiochemical properties of galectin-14. Antagonists may be any compound capable of blocking, inhibiting or otherwise preventing galectin-14 from carrying out its normal biological functions. Antagonists include monoclonal antibodies specific for galectin-14, or parts of galectin-14, and antisense nucleic acids which prevent transcription or translation of galectin-14 genes or mRNA in mammalian cells. Modulation of galectin-14 expression may also be achieved utilising antigens, RNA, ribosomes, DNAzymes, RNA aptamers or antibodies.

Preferably, said agent is an anti-galectin-14 antibody or an anti-galectin-14 antibody.

Said proteinaceous or non-proteinaceous molecule may act either directly or indirectly to modulate the expression of galectin-14 or the activity of galectin-14. Said molecule acts directly if it associates with galectin-14 or galectin-14 to modulate the expression or activity of galectin-14 or galectin-14. Said molecule acts indirectly if it associates with a molecule other than galectin-14 or galectin-14 which other molecule either directly or indirectly modulates the expression or activity of galectin-14 or galectin-14. Accordingly, the method of the present invention encompasses the regulation of galectin-14 or galectin-14 expression or activity via the induction of a cascade of regulatory steps which lead to the regulation of galectin-14 or galectin-14 expression or activity.

In a preferred embodiment of the present invention, the galectin-14, galectin-14 or agent used in the method is linked to an antibody specific for said target cells to enable specific delivery to these cells.

In another aspect there is provided a method of treating a mammal said method comprising administering to said mammal an effective amount of an agent for a time and under conditions sufficient to modulate the expression of galectin-14 or sufficient to modulate the activity of galectin-14 wherein said modulation results in modulation of immune functioning.

In yet another aspect the present invention relates to a method of treating a mammal said method comprising administering to said mammal an effective amount of galectin-14 or galectin-14 for a time and under conditions sufficient to modulate immune functioning.

Reference to “modulating” immune functioning should be understood as a reference to inducing, inhibiting, up-regulating or down-regulating any one or more aspects of immune functioning. Reference to “immune functioning” should be understood to encompass any activity which is associated with the functioning of the immune system. This includes, inter alia, proliferation and differentiation of specific immune cells (e.g. T or B cells) or non-specific immune cells (e.g. eosinophils), cellular apoptosis, cytokine production, activation of immune cells and the induction of immune response effector functions. In a preferred embodiment, the subject immune functioning is an inflammatory response and even more particularly a Th-2 type inflammatory response.

A further aspect of the present invention relates to the use of the invention in relation to the therapeutic or prophylactic treatment of mammalian disease conditions characterised by an aberrant, unwanted or otherwise inappropriate inflammatory response. Reference to the treatment or prophylaxis of an “inflammatory response” should be understood as a reference to the treatment of any disease or other condition, the symptoms, cause or side effects of which include inflammation or the occurrence of one or more components or steps of an inflammatory pathway but which may not have yet resulted in inflammation occurring. This includes, for example, inflammatory conditions which occur as a side effect of a treatment regime for an unrelated disease condition. Examples of inflammatory conditions include, but are not limited to, allergic conditions, bronchitis, bronchilitis, acute respiratory distress syndrome, cystic fibrosis or hayfever. Preferably, said condition is an allergic condition and said inflammatory response is a Th-2 type inflammatory response.

Reference to an “aberrant, unwanted or otherwise inappropriate” inflammatory response should be understood as a reference to excessive inflammation, ineffective inflammation or to physiologically normal inflammation which is inappropriate in that it is unwanted or insufficient.

Accordingly, another aspect of the present invention relates to a method for treatment and/or prophylaxis of a condition characterised by an aberrant, unwanted or otherwise inappropriate inflammatory response in a mammal said method comprising administering to said mammal an effective amount of an agent for a time and under conditions sufficient to modulate the expression of galectin-14 or sufficient to modulate the activity of galectin-14 wherein said modulation results in modulation of said inflammatory response.

In another aspect the present invention relates to a method for the treatment and/or prophylaxis of a condition characterised by an aberrant, unwanted or otherwise inappropriate inflammatory response in a mammal said method comprising administering to said mammal an effective amount of galectin-14 or galectin-14 for a time and under conditions sufficient to modulate said inflammatory response.

In the context of these aspects of the present invention, reference to “modulation” should be understood as a reference to the induction, inhibiting, up-regulation or down-regulation of any one or more aspects of an inflammatory response. Although the method of the present invention is preferably directed to down-regulating an inflammatory response, there may be some situations in which up-regulation of an inflammatory response is desired, for example, to increase protection against infectious agents such as parasites. In accordance with this preferred aspect of the invention, the method of the present invention facilitates the subject inflammation being reduced, retarded or otherwise inhibited. Reference to “reduced, retarded or otherwise inhibited” should be understood as a reference to inducing or facilitating the partial or complete inhibition of any one or more components of the inflammatory response. In this regard, it should be understood that an inflammatory response is a complex response comprising numerous physiological events which often occur simultaneously. Said inhibition may occur by either direct or indirect mechanisms. For example, the inhibition of galectin-14 may result in the inhibition of chemotaxes directly. Alternatively, the inhibition of galectin-14 may directly result in the inhibition of the synthesis of a non-galectin-14 molecule which, in turn results in the inhibition of one or more aspects of the inflammatory response.

In a preferred embodiment, said condition is an allergic condition and said inflammatory response is a Th-2 type inflammatory response which is down-regulated via the down-regulation of galectin-14 functional activity.

In yet another aspect, the present invention provides a method for the treatment and/or prophylaxis of an allergic condition, said method comprising administering to said mammal an effective amount of an agent for a time and under conditions sufficient to down-regulate the expression of galectin-14 or sufficient to down-regulate the activity of galectin-14 for a time and under conditions sufficient to down-regulate a Th-2 type inflammatory response.

As detailed hereinbefore, to the extent that it is an object to decrease an inflammatory response by decreasing galectin-14 functional activity, said agent is preferably an antibody (such as the monoclonal antibody secreted by hybridoma clone 1.2 disclosed herein). Without limiting the present invention to any one theory or mode of action, the administration of an antibody directed to galectin-14 would act to bind unbound galectin-14 thereby decreasing the concentration or galectin-14 which is free to interact with its ligand in order to up-regulate inflammation. Accordingly, the use of such an antibody would effectively down-regulate the inflammatory response.

In this regard, an “effective amount” means an amount necessary to at least partly attain the desired effect, or to delay the onset of, inhibit the progression of, or halt altogether, the onset of progression of the particular condition being treated. Such amounts will depend, of course, on the particular conditions being treated, the severity of the condition and individual patient parameters including age, physical conditions, size, weight and concurrent treatment. These factors are well known of those of ordinary skill in the art and can be addressed with no more than routine experimentation. It is preferred generally that a maximum dose be used, that is, the highest safe dose according to sound medical judgement. It will be understood by those of ordinary skill in the art, however, that a lower dose or tolerable dose may be administered for medical reasons, psychological reasons or for virtually any other reasons.

Reference herein to “treatment” and “prophylaxis” is to be considered in its broadest context. The term “treatment” does not necessarily imply that a mammal is treated until total recovery. Similarly, “prophylaxis” does not necessarily mean that the subject will not eventually contract a disease condition. Accordingly, treatment and prophylaxis include amelioration of the symptoms of a particular condition or prevent or otherwise reducing the risk of developing a particular condition. The term “prophylaxis” may be considered as reducing the severity of onset of a particular condition. “Treatment” may also reduce the severity of an existing condition or the frequency of acute attacks (for example, reducing the frequency of acute asthma attacks).

The subject of the treatment or prophylaxis is generally a mammal such as but not limited to human, primate, livestock animal (e.g. sheep, cow, horse, donkey, pig) companion animal (e.g. dog, cat) laboratory test animal (e.g. mouse, rabbit, rat, guinea pig, hamster) captive wild animal (e.g. fox, deer). Preferably the mammal is a human or primate. Most preferably the mammal is a human. Although the present invention is exemplified utilising an ovine model, this is not intended as a limitation on the application of the method of the present invention to other species, in particular, humans.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant.

Yet another aspect of the present invention relates to the use of an agent capable of modulating the expression of galectin-14 or modulating the activity of galectin-14 in the manufacture of a medicament for the modulation of an inflammatory response.

Preferably, up-regulation of said inflammatory response is achieved by up-regulating galectin-14 functioning and down-regulating said response is achieved by down-regulating galectin-14 functioning.

Preferably, said agent is an anti-galectin-14 antibody.

A further aspect of the present invention relates to the use of galectin-14 or galectin-14 in the manufacture of a medicament for the modulation of an inflammatory response.

Still yet another aspect of the present invention relates to agents for use in modulating galectin-14 expression or galectin-14 activity wherein said modulation results in modulation of an inflammatory response.

Another aspect of the present invention relates to galectin-14 or galectin-14 for use in modulating an inflammatory response.

In yet another aspect of the present invention, and without limiting the present invention in any way, galectin-14 expresses a Bcl-2-like motif. Accordingly, it is thought that this molecule may regulate apoptosis via this domain. In particular, galectin-14 may heterodimerise with Bcl-2 through this motif to inhibit Fas-antibody-mediated apoptosis.

Accordingly, yet another aspect of the present invention is directed to a method of modulating aberrant, unwanted or otherwise inappropriate cellular apoptosis in a mammal, said method comprising administering to said mammal an effective amount of an agent for a time and under conditions sufficient to modulate the expression of galectin-14 or sufficient to modulate the activity of galectin-14 wherein up-regulating said expression or activity leads to down-regulation of cellular apoptosis and down-regulating said expression or activity leads to up-regulation of cellular apoptosis.

Still another aspect of the present invention is directed to a method for the treatment and/or prophylaxis of a condition characterised by aberrant, unwanted or otherwise inappropriate cellular apoptosis in a mammal, said method comprising administering to said mammal an effective amount of an agent for a time and under conditions sufficient to modulate the expression of galectin-14 or sufficient to modulate the activity of galectin-14 wherein up-regulating said expression or activity leads to down-regulation of cellular apoptosis and down-regulating said expression or activity leads to up-regulation of cellular apoptosis.

Reference to “aberrant, unwanted or otherwise inappropriate” cellular apoptosis should be understood to have a meaning analogous to that defined in relation to “aberrant, unwanted or otherwise inappropriate” inflammatory response in that the subject apoptosis is excessive, ineffective or physiologically normal but nevertheless inappropriate in that it is unwanted or insufficient.

Yet another aspect of the present invention relates to the use of an agent capable of modulating the expression of galectin-14 or modulating the activity of galectin-14 in the manufacture of a medicament for the modulation of apoptosis.

A further aspect of the present invention relates to the use of galectin-14 or galectin-14 in the manufacture of a medicament for the modulation of apoptosis.

Still yet another aspect of the present invention relates to agents for use in modulating galectin-14 expression or galectin-14 activity wherein said modulation results in modulation of apoptosis.

Another aspect of the present invention relates to galectin-14 or galectin-14 for use in modulating apoptosis.

Administration of the agent, galectin-14 or functional equivalent, derivative, homologue, analogue or mimetic thereof, or galectin-14 nucleic acid molecule (herein referred to as “modulatory agent”), in the form of a pharmaceutical composition, may be performed by any convenient means. The modulatory agent of the pharmaceutical composition contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation. The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

In accordance with these methods, the modulatory agent defined in accordance with the present invention may be coadministered with one or more other compounds or molecules. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules, These molecules may be administered in any order.

In yet another further aspect the present invention contemplates a pharmaceutical composition comprising galectin-14, galectin-14 or an agent capable of modulating galectin-14 expression or galectin-14 activity together with one or more pharmaceutically acceptable carriers and/or diluents. Galectin-14, galectin-14 or said agent are referred to as the active ingredients.

The pharmaceutical forms suitable for injectable use include sterile aqueous solutions (where water soluble) and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as licithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations. Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active ingredient is compounded for convenient and effective administration in effective amounts with a suitable pharmaceutically acceptable carrier in dosage unit form as hereinbefore disclosed. A unit dosage form can, for example, contain the principal active compound in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

The pharmaceutical composition may also comprise genetic molecules such as a vector capable of transfecting target cells where the vector carries a nucleic acid molecule capable of modulating galectin-14 expression or galectin-14 activity. The vector may be, for example, a viral vector. The present invention should be understood to extend to the use of such vectors in gene therapy.

Screening for the modulatory agents hereinbefore defined can be achieved by any one of several suitable methods including, but in no way limited to, contacting a cell comprising the galectin-14 gene or functional equivalent or derivative thereof with an agent and screening for the modulation of galectin-14 protein production or functional activity, modulation of the expression of a nucleic acid molecule encoding galectin-14 or modulation of the activity or expression of a downstream galectin-14 target. Detecting such modulation can be achieved utilising techniques such as Western blotting, electrophoretic mobility shift assays and/or the readout of reporters of galectin-14 activity such as luciferases, CAT and the like.

It should be understood that the galectin-14 gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate galectin-14 activity, at either the nucleic acid or expression product levels, or the gene may require activation—thereby providing a model useful for, inter alia, screening for agents which up regulate galectin-14 expression. Further, to the extent that an galectin-14 nucleic acid molecule is transfected into a cell, that molecule may comprise the entire galectin-14 gene or it may merely comprise a portion of the gene such as the portion which regulates expression of the galectin-14 product. For example, the galectin-14 promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively.

In another example, the subject of detection could be a downstream galectin-14 regulatory target, rather than galectin-14 itself or the reporter molecule ligated to the galectin-14 promoter or the reporter gene ligated to the promoter of the gene which galectin-14 regulates. Yet another example includes galectin-14 binding sites ligated to a minimal reporter. For example, modulation of galectin-14 activity can be detected by screening for the modulation of the functional activity in an eosinophil. This is an example of an indirect system where modulation of galectin-14 expression, per se, is not the subject of detection. Rather, modulation of the molecules which galectin-14 regulates the expression of, are monitored. Where the cell which is the subject of the screening system is an epithelial cell, modulation of galectin-14 expression could be detected by screening for modulation of the proliferative activity of that cell.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the galectin-14 nucleic acid molecule or expression product itself or which modulate the expression of an upstream molecule, which upstream molecule subsequently modulates galectin-14 expression or expression product activity. Accordingly, these methods provide a mechanism of detecting agents which either directly or indirectly modulate galectin-14 expression and/or activity.

Accordingly, another aspect of the present invention provides a method for detecting an agent capable of modulating the function of galectin-14 or its functional equivalent or derivative thereof said method comprising contacting a cell or extract thereof containing said galectin-14 or its functional equivalent or derivative with a putative agent and detecting an altered expression phenotype associated with said galectin-14 or its functional equivalent or derivative.

Reference to “galectin-14” should be understood as a reference to either the galectin-14 expression product or a nucleic acid molecule encoding galectin-14. It should also be understood as a reference to a portion or fragment of the galectin-14 molecule such as the regulatory region of the galectin-14 nucleic acid molecule. Alternatively, the molecule may comprise the binding portion of the galectin-14 expression product. In this regard, the galectin-14 nucleic acid molecule and/or expression product is expressed in a cell. The cell may be a host cell which has been transfected with the galectin-14 nucleic acid molecule or it may be a cell, such as an eosinophil, which naturally contains the galectin-14 gene. Reference to “extraction thereof” should be understood as a reference to a cell free transcription system.

Reference to detecting an “altered expression phenotype associated with said galectin-14” should be understood as the detection of cellular changes associated with modulation of the activity of galectin-14. These may be detectable for example as intracellular changes or changes observable extracellularly. For example, this includes, but is not limited to, detecting changes in expression product levels, or, to the extent that the galectin-14 regulatory region is ligated to a reporter molecule such as luciferase or CAT, detecting changes in reporter molecule expression. Alternatively, this screening system may be established to detect changes in the expression of downstream molecules which are regulated by the galectin-14 expression product.

As detailed earlier, the method of this aspect of the present invention should be understood to extend to screening for agents which modulate the expression of galectin-14 either directly or indirectly. An example of indirect modulation of galectin-14 would be modulation of the expression of a first nucleic acid molecule, which first nucleic acid molecule expression product modulates the expression of a nucleic acid molecule encoding galectin-14 or functional equivalent or derivative thereof.

In yet another aspect the present invention provides a method for detecting an agent capable of binding or otherwise associating with a galectin-14 binding site or functional equivalent or derivative thereof said method comprising contacting a cell containing said galectin-14 binding site or functional equivalent or derivative thereof with a putative agent and detecting an altered expression phenotype associated with modulation of the function of galectin-14 or its functional equivalent or derivative.

In still another aspect the present invention provides a method for detecting an agent capable of binding or otherwise associating with a galectin-14 binding site or functional equivalent or derivative thereof said method comprising contacting a cell containing said galectin-14 binding site or functional equivalent or derivative thereof with a putative agent and detecting an altered expression phenotype associated with modulation of the function of galectin-14 or its functional equivalent or derivative.

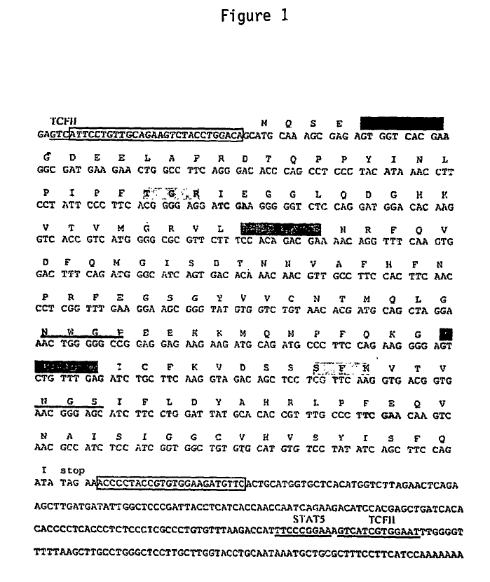

Reference to a galectin-14 or galectin-14 “binding site” should be understood as a reference to the amino acid and nucleic acid regions, respectively, which interact with other proteinaceous or non-proteinaceous molecules. For example, and without limiting the present invention in any way, it has been determined that amino acid residues His⁷⁴, Asn⁷⁶, Arg⁷⁸, Asn⁸⁸, Trp⁹⁵ and Glu⁹⁸ of <400>1 define a site of sugar recognition and binding (refer FIG. 1). Three casein kinase II binding sites and two protein kinase C binding sites have also been identified and are indicated in FIG. 1. In another example, two transcription factor binding sites (STAT5 and TCF11) have been identified and are indicated in FIG. 1.

In addition to screening for agents which modulate the interaction of galectin-14 protein and nucleic acid sequences with other molecules, utilising function based assays of the type described above, the identification of galectin-14 binding sites, such as the carbohydrate recognition residues, facilitates the screening, analysis, rational design and/or modification of agents for modulating the interaction of galectin-14 with its ligands based on analysis of the physical interaction of a putative agent or lead compound with the subject binding site.

In particular, knowledge of the nature and location of the carbohydrate, protein kinase C and casein kinase II binding sites now facilitates analysis of the tertiary structure of galectin-14, in terms of the structure of the binding site, by techniques such as X-ray crystallography.

Accordingly, another aspect of the present invention is directed to a method for analysing, designing and/or modifying an agent capable of interacting with the carbohydrate, protein kinase C and/or casein, kinase II binding site of galectin-14 or derivative thereof and modulating at least one functional activity associated with said galectin-14 said method comprising contacting said galectin-14 or derivative thereof with a putative agent and assessing the degree of interactive complementarity of said agent with said binding site.

Preferably said galectin-14 binding site is defined by one or more of:
(i) the amino acids H⁷⁴, N⁷⁶, R⁷⁸, N⁸⁸, W⁹⁵ and E⁹⁸ of <400>1;
(ii) the amino acid sequence SGHE (position numbers 5-8 of <400>1), STDE (position numbers 51-54 of <400>1), or SLFE (position numbers 110-113 of <400>1);
(iii) the amino acid sequence TGR (position numbers 30-32 of <400>1) or SFK (position numbers 122-124 of <4001>1).

It should be understood that the galectin-14 which is contacted with the putative agent for evaluation of interactive complementarity may be recombinantly produced. However, it should also be understood that the subject galectin-14 may take the form of an image based on the binding site structure which has been elucidated, such as an electron density map, molecular models (including, but not limited to, stick, ball and stick, space filling or surface representation models) or other digital or non-digital surface representation models or image, which facilitates the analysis of galectin-14 site: agent interactions utilising techniques and software which would be known to those of skill in the art. For example, interaction analyses can be performed utilising techniques such as Biacore real-time analysis of on and off-rates and dissociation constants for binding of ligands (Gardsvoll et al, (1999) J. Biol. Chem. 274(53):37995-38003; Hoyer-Hansen et al, (1997) FEBS Lett 420(1):79-85; Ploug et al., (1998) Biochemistry 37(47):16494-16505; Ploug et al. (1994) Biochemistry 33(30):8991-8997; Ploug et al. (1995) Biochemistry 34(39):12524-12534; Ploug et al. (1998) Biochemistry 37(110):3612-3522) and NMR perturbation studies (Stephens et al, 1992).

Reference to “assessing the degree of interactive complementarity” of an agent with the subject galectin-14 binding site should be understood as a reference to elucidating any feature of interest including, but not limited to, the nature and/or degree of interaction between the subject galectin-14 binding site and an-agent of interest. As detailed above, any suitable technique can be utilised. Such techniques would be known to the person of skill in the art and can be utilised in this regard. In terms of the nature of the subject interaction, it may be desirable to assess the types of interactive mechanisms which occur between specific residues of any given agent and those of the galectin-14 binding site (for example, peptide bonding or formation of hydrogen bonds, ionic bonds, van der Waals forces, etc.) and/or their relative strengths. It may also be desirable to assess the degree of interaction which occurs between an agent of interest and the subject galectin-14 binding site. For example, by analysing the location of actual sites of interaction between the subject agent and galectin-14 binding site it is possible to determine the quality of fit of the agent into this region of the galectin-14 binding site and the relative strength and stability of that binding interaction. For example, if it is the object that galectin-14 binding site functioning be blocked, an agent which interacts with the galectin-14 binding site such that it blocks or otherwise hinders (for example, sterically hinders or chemically or electrostatically repels) the interaction of interest will be sought. The form of association which is required in relation to modulating galectin-14 functioning may not involve the formation of any chemical interactive bonding mechanism, as this is traditionally understood, but may involve a non-bonding mechanism such as the proximal location of a region of the agent relative to the subject binding region of the galectin-14 binding site, for example, to effect steric hindrance with respect to the binding of an activating molecule. Where the interaction takes the form of hindrance or the creation of other repulsive forces, this should nevertheless be understood as a form of “interaction” despite the lack of formation of any of the traditional forms of bonding mechanisms.

It should also be understood that the galectin-14 binding site which is utilised either in a physical form or as an image, as hereinbefore discussed, to assess the interactive complementarity of a putative agent may be a naturally occurring form of the galectin-14 binding site or it may be a derivative, homologue, analogue, mutant, fragment or equivalent thereof. The derivative, homologue, analogue, mutant, fragment or equivalent thereof may take either a physical or non-physical (such as an image) form.

The determination of galectin-14 binding regions facilitates determination of the three dimensional structure of the galectin-14 binding site and the identification and/or rational modification and design of agents which can be used to modulate galectin-14 functioning.

Without limiting the application of the present invention in any way, the method of the present invention facilitates the analysis, design and/or modification of agents capable of interacting with the galectin-14 binding site. In this regard, reference to “analysis, design and/or modification” of an agent should be understood in its broadest sense to include:
(i) Randomly screening (for example, utilising routine high-throughput screening technology) to identify agents which exhibit some modulatory capacity with respect to galectin-14 functional activity and then analysing the precise nature and magnitude of the agent's modulatory capacity utilising the method of this aspect of the present invention. In this regard, existing crystals could be soaked with said agents or co-crystalisation could be performed. A combination of modelling and synthetic modification of the local compound together with mutagenesis of the galectin-14 binding site could then be performed for example. In screening for agents which may modulate activity, standard methods of phage display and also combinatorial chemistry may be utilised (Goodson, et al. (1994) Proc. Natl. Acad. Sci. USA 91(15):7129-7133; Terrett, N. (2000) Drug Discovery Today 5(5):211-212). Such interaction studies can also be furthered utilising techniques such as the Biacore analysis and NMR perturbation studies. Such agents are often commonly referred to as “lead” agents in terms of the random screening of proteinaceous or non-proteinaceous molecules for their capacity to function either agonistically or antagonistically. Further, for example, binding affinity and specificity could be enhanced by modifying lead agents to maximise interactions with the galectin-14 binding site. Such analyses would facilitate the selection of agents which are the most suitable for a given purpose. In this way, the selection step is based not only on in vitro data but also on a technical analysis of sites of agent: galectin-14 interaction in terms of their frequency, stability and suitability for a given purpose. For example, such analysis may reveal that what appears to be an acceptable in vitro activity in respect of a randomly identified agent is in fact induced by a highly unstable interaction due to the presence of proximally located agent: galectin-14 sites which exhibit significant repulsive forces thereby de-stabilising the overall interaction between the agent and the galectin-14. This would then facilitate the selection of another prospective lead compound, exhibiting an equivalent degree of in vitro activity, but which agent does not, upon further analysis, involve the existence of such de-stabilising repulsive forces.
Screening for the modulatory agents herein defined can be achieved by any one of several suitable methods, including in silico methods, which would be well known to those of skill in the art and which are, for example, routinely used to randomly screen proteinaceous and non-proteinaceous molecules for the purpose of identifying lead compounds.
These methods provide a mechanism for performing high throughput screening of putative modulatory agents such as the proteinaceous or non-proteinaceous agents comprising synthetic, recombinant, chemical and natural libraries.
(ii) The candidate or lead agent (for example, the agent identified in accordance with the methodology described in relation to point (i)) could be modified in order to maximise desired interactions (for example, binding affinity to specificity) with the galectin-14 and to minimise undesirable interactions (such as repulsive or otherwise de-stabilising interactions).
Methods of modification of a candidate or lead agent in accordance with the purpose as defined herein would be well known to those of skill in the art. For example, a molecular replacement program such as Amore (Navaza, 1994) may be utilised in this regard. The method of the present invention also facilitates the mutagenesis of known signal inducing agents in order to ablate or improve signalling activity.
(iii) In addition to analysing fit and/or structurally modifying existing molecules, the method of the present invention also facilitates the rational design and synthesis of an agent, such as an agonistic or antagonistic agent, based on theoretically modelling an agent exhibiting the desired sphingosine kinase binding site interactive structural features followed by the synthesis and testing of the subject agent.

It should be understood that any one or more of applications (i)-(iii) above, may be utilised in identifying a particular agent.

Preferably, said agent is an anti-galectin-14 antibody.

In a related aspect, the present invention should be understood to extend to the agents identified utilising any of the methods hereinbefore defined. In this regard, reference to an agent should be understood as a reference to any proteinaceous or non-proteinaceous molecule which modulates at least one galectin-14 mediated functional activity.

Still another aspect of the present invention is directed to antibodies to galectin-14 including catalytic antibodies. Such antibodies may be monoclonal or polyclonal and may be selected from naturally occurring antibodies to galectin-14 or may be specifically raised to galectin-14. In the case of the latter, galectin-14 may first need to be associated with a carrier molecule. The antibodies and/or recombinant galectin-14 of the present invention are particularly useful as therapeutic or diagnostic agents. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool.

For example, galectin-14 can be used to screen for naturally occurring antibodies to galectin-14.

For example, specific antibodies can be used to screen for galectin-14 proteins. The latter would be important, for example, as a means for screening for levels of galectin-14 in a cell extract or other biological fluid or purifying galectin-14 made by recombinant means from culture supernatant fluid. Techniques for the assays contemplated herein are known in the art and include, for example, sandwich assays, ELISA and flow cytometry.

It is within the scope of this invention to include any second antibodies (monoclonal, polyclonal or fragments of antibodies) directed to the first mentioned antibodies discussed above. Both the first and second antibodies may be used in detection assays or a first antibody may be used with a commercially available anti-immunoglobulin antibody. An antibody as contemplated herein includes any antibody specific to any region of galectin-14.

Both polyclonal and monoclonal antibodies are obtainable by immunization with the protein or peptide derivatives and either type is utilizable for immunoassays. The methods of obtaining both types of sera are well known in the art. Polyclonal sera are less preferred but are relatively easily prepared by injection of a suitable laboratory animal with an effective amount of galectin-14, or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example Douillard and Hoffman, Basic Facts about Hybridomas, in Compendium of Immunology Vol II, ed. by Schwartz, 1981; Kohler and Milstein, Nature 256: 495-499, 1975; European Journal of Immunology 6: 511-519, 1976).

Preferably said antibody is a monoclonal antibody produced by clone 1.2 or derivative, homologue, analogue, chemical equivalent or mimetic of said antibody.

In another aspect of the present invention, the molecules of the present invention are also useful as screening targets for use in applications such as the diagnosis of disorders which are regulated by galectin-14.

Screening for galectin-14 or galectin-14 in a biological sample can be performed by any one of a number of suitable methods which are well known to those skilled in the art.

Examples of suitable methods include, but are not limited to, in situ hybridisation of biopsy sections to detect mRNA transcript or DNA, Northern blotting, RT-PCR of specimens isolated from tissue biopsies or bodily fluid samples (such as blood), antibody screening of tissue sections or bodily fluid samples.

To the extent that antibody based methods of diagnosis are used, the presence of galectin-14 or galectin-14 may be determined in a number of ways such as by Western blotting, ELISA or flow cytometry procedures. These, of course, include both single-site and two-site or “sandwich” assays of the non-competitive types, as well as in the traditional competitive binding assays. These assays also include direct binding of a labelled antibody to a target.

Sandwich assays are among the most useful and commonly used assays and are favoured for use in the present invention. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent. In accordance with the present invention the sample is one which might contain galectin-14 including cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

In the typical forward sandwich assay, a first antibody having specificity for the galectin-14 or antigenic parts thereof, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes) and under suitable conditions (e.g. 25° C.) to allow binding of any subunit present in the antibody. Following the incubation period, the antibody subunit solid phase is washed and dried and incubated with a second antibody specific for a portion of the hapten. The second antibody is linked to a reporter molecule which is used to indicate the binding of the second antibody to the hapten. An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells on latex beads, and the like.

Alternately, fluorescent compounds, such as fluorecein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength the fluorescence observed indicates the presence of the hapten of interest. Immunofluorescene and EIA techniques are both very well established in the art and are particularly preferred for the present method. However, other reporter molecules, such as radioisotope, chemiluminescent or bioluminescent molecules, may also be employed.

Further features of the present invention are more fully described in the following non-limiting examples.

### EXAMPLE 1

### Isolation and Characterisation of Galectin-14 in H. Contortus Primed Animals

Materials And Methods

Abomasum Tissue samples

Nematode free adult Merino sheep (wethers) were immunised and challenged with H. contortus L3 larvae as described previously (Dunphy, J. L., Balic, A., Barcham, G. J., Horvath, A. J., Nash, A. D. and Meeusen, E. N. T. (2000) J. Biol. Chem. 275:32106-32113). Sheep challenged with PBS alone were used as controls. Tissues from 3 month old worn free sheep after primary H. contortus larvae infection were also collected (Dunphy et al. 2000 supra). Control lambs were uninfected. All sheep were sacrificed 2, 3 or 5 days post-challenge and samples of abomasum collected for histology and RNA preparation.

Separate sheep were immunised by repeated infections of 5,000-10,000 H. contortus L3 larvae. Three months after the last infection they were challenged with 50,000 L3 and sacrificed 10 days later. Mucus was collected from the abomasum of these sheep for Western blot analysis, by gently scraping mucus off the surface of the mucosa, diluting it approximately ⅓ with phosphate buffered saline (PBS), centrifuging it to remove cellular debris, and collecting the supernatant.

Mammary Tissue Samples

Mature non-lactating Merino ewes were primed by weekly intramammary infusions of H. contortus L3 larvae for 4-5 weeks (Greenhalgh et al. 1996 supra, Rainbird, M. A., Macmillan, D. and Meeusen, E. N. (1998) Parasite Immunol. 20(2):93-103; Dunphy et al. 2000 supra). The ewes were then rested for 3-4 weeks before being challenged by intramammary infusion of ˜5×10³ H. contortus L3 larvae. Other ewes received only one intramammary infusion of H. contortus L3 larvae, or were primed but not challenged. Control sheep received a single intramammary infusion of sterile saline alone. Sheep were sacrificed 2-4 days post-challenge and tissues collected for RNA extraction and histology (Dunphy et al. 2000 supra).

Ewes orally infected with ˜80 metacercariae of the liver fluke Fasciola hepatica 2-4 months previously, also received one intramammary infusion of H. contortus larvae to induce unprimed eosinophil-rich leukocyte infiltration (Rainbird et al. 1998 supra).

Lung Tissue Samples

Sheep were primed by three subcutaneous injections of 50 μg of solubilised house dust mite extract (Dermatophagiodes pteronyssinus; CSL, Melbourne) in saline with aluminium hydroxide as adjuvant (1:1). Two-three weeks later they were challenged with 1 mg of solubilised house dust mite extract (HDME) in saline, in the lower left lung lobe using a bronchoscope. Sheep were sacrificed and lung and BAL samples collected 48 hours post-challenge for histology and RNA preparation. Control sheep were not primed and were challenged with saline alone.

Leukocyte Preparations

Mammary lavage fluid (MAL) of H. contortus primed and challenged, or liver fluke infected and H. contortus challenged ewes, were collected 2-5 days post-challenge as described previously (Rainbird et al. 1998 supra, Dunphy et al. 2000 supra). Some of the “unprimed” leukocytes collected from liver fluke infected, H. contortus challenged sheep MAL were cultured for 5 or 24 hours with 100 ng/ml of human recombinant interleukin 5 (IL-5; Genzyme Diagnostics, Cambridge, Mass.) before RNA extraction. The proportion of eosinophils in the leukocyte suspensions, as determined by Giesma stained cytospots, varied from 52-92%. Monocytes and lymphocytes constituted the remainder of the cell population (5-32% and 3-16% respectively).

Multiple intramammary infusions of 1 mg HDME (solublized in saline) also resulted in a very high proportion of eosinophils in the MAL cell populations collected 2 days post challenge (75-90%).

Separate sheep received a single intramammary infusion of LPS and MAL cells were collected (as described above) at 24 h and 5 days, which results in an initial influx of neutrophils (24 h) followed by macrophage infiltration (5 days).

Leukocytes were also collected from the bronchoalveolar lavage fluid (BAL) of sheep given HDME lung challenge. Control BAL cells were collected from unsensitised, saline challenge sheep. The sheep were sacrificed and the lungs and heart removed from the chest cavity. The left and right BAL was collected separately by occluding the entrance to one lung lobe with a Foley catheter, and then pouring 250 ml of saline down the trachea, massaging the lung, draining the fluid and repeating once before removing the catheter and repeating for the other lung lobe. The saline/BAL fluid mix was centrifuged, the pelleted cells treated with TAC (0.17 M Tris/0.16 M NH₄Cl pH 7.2) to remove contaminating red blood cells, and the cells washed three times before being used for RNA preparation and cytospot analysis. Cytospot analysis indicated that eosinophils represented 5-38% of cells in HDME treated BAL, while no eosinophils detected in the BAL of control sheep.

Proteins were extracted from isolated leukocytes by pelleting the cells, lysing them by a freeze/thaw cycle, resuspending the pellet in saline, and then separating soluble proteins from cellular debris by centrifugation. Both soluble proteins in the supernatant and those trapped in the pellet were analysed.

RNA Preparation

Total RNA was purified from tissues using a standard guanidinium thiocyanate, phenol/chloroform extraction (Chomczynski, P. and Sacchi, N. (1987) Anal. Biochem. 162:156-159). 0.1-1 g of tissue, 0.5-1 ml of packed larvae, or ˜1×10⁸ cells were used.

Low Stringency RT-PCR

To amplify cDNA clones from MAL cells that were differentially expressed by fresh and cultured cells, low stringency RT-PCR was performed using the display PROFILE kit from Display Systems Biotech (Integrated Sciences, Sydney Australia) as described in the kit manual (version 2.0). Total RNA from MAL leukocytes of H. contortus mammary challenged sheep was used as the template. cDNA synthesis was performed to produce double-stranded cDNA which was digested with the restriction enzyme TaqI to provide sites for the ligation of adaptors. The adaptor sequences were then used as PCR primers with only three nucleotides specific for the cDNA. The PCR primer that resulted in amplification of the partial galectin-14 cDNA was DisplayPROBEsEu4 (see Table 1). The PCR incorporated ³³P-dATP and the PCR products were visualised by autoradiography after separation of denaturing polyacrylamide gels (6% polyacrylamide, 8.3M urea). PCR products of interest were re-amplified and subcloned into pGEM-Teasy (Promega) before being sequenced using the BIG DYE terminator mix (Perkin Elmer Applied Biosystems).

Construction and Screening of a MAL cDNA Library

The SMART cDNA library construction kit (Clontech) was used as instructed by the manufacturer to prepare a cDNA library representing mRNA expressed in an eosinophil rich leukocyte population from H. contortus challenged MAL. ˜1 μg of total RNA extracted from MAL cells was used. The cDNA was amplified by PCR using the Advantage cDNA polymerase mix from Clontech that is optimised for the amplification of large cDNA. The cDNA was size fractionated with the Chromospin 400 columns, fractions 5-8 were pooled and ligated to λTriplEx2 SfiI digested arms using T4 DNA ligase (Promega). These fractions were chosen since the galectin-14 mRNA transcript was estimated to be ˜800 bp from Northern blot hybridisation. The ligation mixes were packaged by the Packagene phage packaging system from Promega (as described in the Packagene manual). LE392 cells were infected with the pTriplEx2 phage and the plaques screened with the galectin-14 partial RT-PCR cDNA Klenow probe as instructed in the Packagene manual. The hybridisation and wash conditions used were the same as for Northern blot analysis (see below). At least 1×10⁶ pfu were used for each primary screen.

Once individual plaques of interest were isolated in tertiary screens, the λTriplEx2 phage was converted into the pTriplEx2 plasmid by infecting BM25.8 cells and inducing their Cre-recombinase at 31° C. (as instructed in the SMART cDNA manual). The cDNA were then sequenced using the BIG DYE terminator mix (Perkin Elmer) and the 5′ sequencing primer of pTriplEx2 (supplied by Invitrogen).

Amplification of Galectin-14 cDNA, Containing the Full Putative Coding Region

Two RT-PCR primers were designed within the putative 5′ and 3′ untranslated regions (UTRs) of galectin-14 (galectin-145′UTR and galectin-143′UTR, see Table 3). ˜1.25 μg of unprimed MAL total RNA was used as a template for reverse transcriptase. The reaction contained 1.25 μM of 3′ primer, 20 U of Rnaseguard, 10 mM DTT, 1 mM of each dNTP, and 200 U of Superscript II RT in a total volume of 20 μl. 2-10 μl of the RT mix was used as a template for 30 PCR cycles. The PCR used 0.25 μM of each primer, 200 μM of each dNTP and 2.5 U of Taq polymerase in a total volume 100 μl. The 30 PCR cycles utilised a denaturation step of 95° C. for 30 seconds, an annealing temperature of 54° C. for 1 minute, and an extension temperature of 74° C. for 1 minute. An additional denaturation of 5 minutes preceded the 30 cycles, and a prolonged extension of 10 minutes completed the PCR. PCR products were subcloned into pGEM-Teasy (Promega) and sequenced using BIG Dye (Perkin Elmer Applied Biosystems) automated sequencing.

Northern Blot Hybridisations

Northern blots were prepared as described by Fourney, R. M., Mlyakoshi, J., Day III, R. S. and Paterson, M. C. (1988) Focus 10(1):5-6. ˜10 μg of total RNA was transferred to Hybond N+ membranes (Amersham Pharmacia Biotech) by capillary action. Membranes were prehybridised for 4 hours at 42° C. in Church buffer [0.5 M sodium phosphate pH 7.2/1% BSA/7% SDS/2 mM EDTA]. ³²P-labelled cDNA probes were generated from the partial galectin-14 cDNA clone using Klenow and the Giga-prime kit (Bresatec, Adelaide, Australia). Probes were denatured at 95° C. for 5 minutes before hybridising to the membranes in Church buffer overnight at 65° C. The membranes were washed at high stringency in 0.2×SSC/0.1% SDS at 37-42° C. Kodak BioMax MS film Eastman Kodak, Rochester, N.Y., USA) was then exposed to the membranes for 1-24 hours, of Fuji XAR film (Fuji Photo Film, Osaka, Japan) was exposed for 16-64 hours.

Production of Recombinant Galectin-14 in E. coli.

To produce anti-galectin-14 polyclonal serum, a rabbit was immunized three times intramuscularly at monthly intervals with ˜45 μg of recombinant galectin-14 emulsified in, initially, 50% Freund's complete adjuvant and, subsequently, 50% Freunds's incomplete adjuvant (Sigma). Serum was collected 7 days after the final immunization. The specificity and activity of the serum was confirmed by enzyme-linked immunosorbent assay (ELISA).

To produce anti-galectin-14 monoclonal antibodies six female BALB/c mice were given IP injections of ˜5 μg of cleaved and purified recombinant galectin-14 once a month for 3 months, initially in complete Freund's adjuvant and subsequently in incomplete Freunds adjuvant. Spleen cells from immune mice were fused with NS-1 myeloma cells using 50% polyethylene glycol 4000 (Merck, Darmstadt, Germany) as described previously (Galfre, G., Howe, S. C., Milstein, C., Butcher, G. W. and Howard, J. C. (1977) Nature 266:550-552). The supernatants were screened for anti-galectin-14 activity and specificity by ELISA. Positive hybridomas were cloned by limiting dilution at least three times before being converted to DM10 media alone. Ascitis fluid was produced by giving pristine primed BALB/c mice an IP injection of 1×10⁷ hybridoma cells (Harlow and Lane 1988).

Western Blot Analysis

Proteins from recombinant protein preparations, eosinophil rich leukocyte protein preparations, stomach mucus scrapings, or cell-free MAL supernatants, were separated by SDS-PAGE and transferred to 0.45 μM nitrocellulose membrances (MSI, Melbourne, Australia) by electroblotting at 100V for 1 h (Towbin, H., Stachelin, T. & Gordon, J. (1979) Proc. Natl. Acad. Sci. (USA) 76:4350-4354). Membranes were incubated with 1/500 dilution of primary sera for 2 h at room temperature. After incubation with secondary antibody for at least 1 h at room temperature, signals were detected by 1.5 mM 3,3′-diaminobenzidine tetrahydrochloride (DAB; Sigma), or ECL (Amersham Pharmacia Biotech).

Immunohistology

Whenever tissue samples were collected for RNA preparation, samples were also taken for histology. They were fixed in 10% neutral-buffered formalin and processed to parafin. 5 μM tissue sections were cut and stained with haematoxylin and eosin (H&E). The sections were examined under the microscope and the numbers of eosinophils infiltrating the mucosa were noted.

For immunohistology 4-5 μM sections of OCT (Tissue Tek: Miles Inc.) embedded, frozen tissue blocks were cut, dried and fixed in 95% ethanol for 10 min at 4° C. Endogenous peroxidase was quenched by adding DAKO peroxidase blocking reagent. Primary polyclonal serum (diluted 1/500) or undiluted mAb culture supernatants were incubated with the slides for 1-2 h at room temperature in a humidity chamber. Peroxide-conjugated anti-rabbit or anti-mouse antibody was detected by incubating slides with DAB for 10 min at room temperature. The slides were counterstained with hematoxylin and Eosin Y.

Cytospots were also stained as above.

Results

Nucleotide Sequence and Predicted Amino Acid sequence of Galectin-14

Whilst screening for cDNA clones that are differentially expressed in fresh and cultured eosinophil-rich MAL cells, a partial cDNA clone of 325 bp was isolated showing similarity to a potent human eosinophil chemoattractant (ecalectin). Northern blot analysis confirmed that this clone was expressed at relatively high levels by the eosinophil-rich leukocyte populations. Hence an eosinophil-rich MAL cell cDNA library was screened to isolate the full-length clone.

The entire coding region (including the stop codon) is 489 bp and encodes a predicted protein of 162 amino acids (FIG. 1). The 5′ untranslated region is only 35 bp and contains a putative binding site for the transcription factor TCF11. The 3′ untranslated region is approximately 260 nucleotides (not including the poly (A) tail) and contains another putative TCF11 binding site as well as a putative signal transducer and activator of transcription (STAT) 5 binding site (MatInspector V2.2, available at http://www.gsf.de/cgi-bin/matsearch.pl).

A PROSITE database search indicates that the predicted protein contains three potential casein kinase II sites, two putative protein kinase C sites and a possible N-glycosylation site (FIG. 1; Bairoch, A., Bucher, P. and Hofmann, K. (1997) Nucl. Acids Res. 25:217-221). Hydrophobicity plots (obtained from ProScale; http://expasy.proeome.org.au/cgi-bin/protscale) also indicate that the protein contains four short hydrophobic regions.

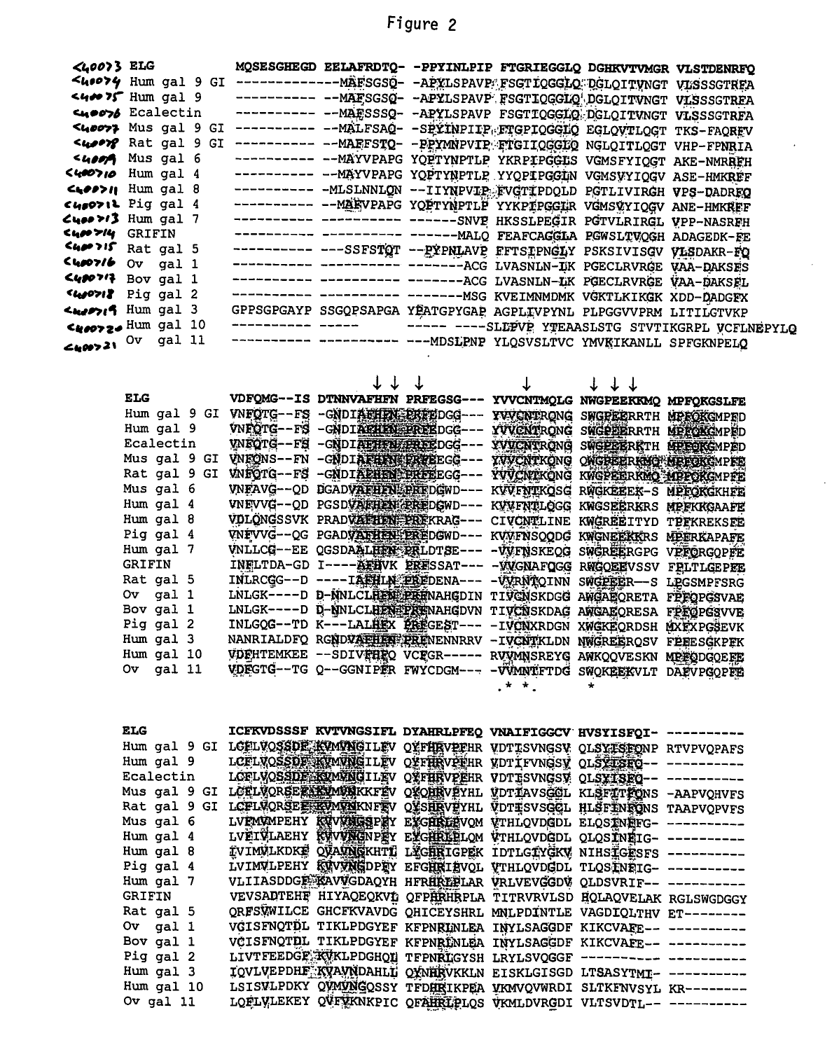

Both a nucleotide Blast database search and a protein Blast search (using the predicted amino acid sequence) indicate that the clone is a novel galectin (see Table 4 and FIG. 2). Galectins are carbohydrate binding proteins and can be classified as proto, tandem-repeat or chimera types. The nucleotide and predicted protein sequences demonstrate most identity to ecalectin/galectin-9 (58% and 57% amino acid identity to rat and human galectin-9 respectively; see Table 4).

Ecalectin/galectin-9 is a tandem-repeat type galectin which contains two carbohydrate recognition domains (CRD; Hirashima 2000 supra). Galectin-14 however contains only one putative CRD which makes up the majority of the protein and therefore is classified as a proto-type galectin. Compared to other known proto-type galectins galectin-14 contains a slightly extended NH2-terminus, however this is still much shorter than that of galectin-3 which is classified as a chimera galectin due to its extended NH2-terminus that is composed of a repetitive proline and glycine rich sequence.

Galectin-14 is expected to bind β-galactosidases like most galectins given that it contains six of the seven residues reported to be important for sugar recognition and bindng by galectin-1 (Hirabayashi and Kasai 1994 supra, Ahmed et al. 1996 supra). Additionally, the amino acid alteration detected in one of these seven residues is a conservative change (R to K₁₀₀; FIG. 2).

Like many other galectins, galectin-14 also contains a Bcl-2 like motif within the CRD which suggests it may regulate apoptosis (FIG. 1).

Northern Blot Analysis of mRNA Levels

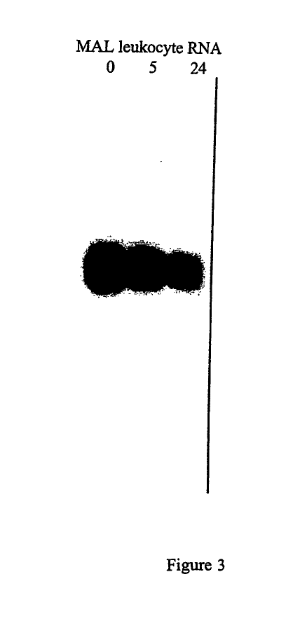

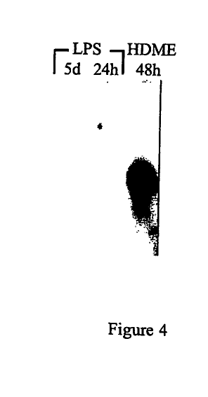

Northern blot analysis has detected relatively high levels of ELF mRNA in eosinophil-rich leukocyte populations that have migrated into the mammary lavage (MAL) in response to intramammary infusions of H. contortus larvae or HDME (FIGS. 3 and 4 respectively). However, expression was not detected in whole normal of H. contortus sensitised mammary tissue. Galectin-14 mRNA levels in MAL cell samples appear to fall after culture (FIG. 3). Galectin-14 mRNA was not detected in monocyte or neutrophil rich MAL leukocyte populations induced by LPS intramammary infusions, indicating that the gene is expressed by eosinophils and not other leukocyte populations (FIG. 4).

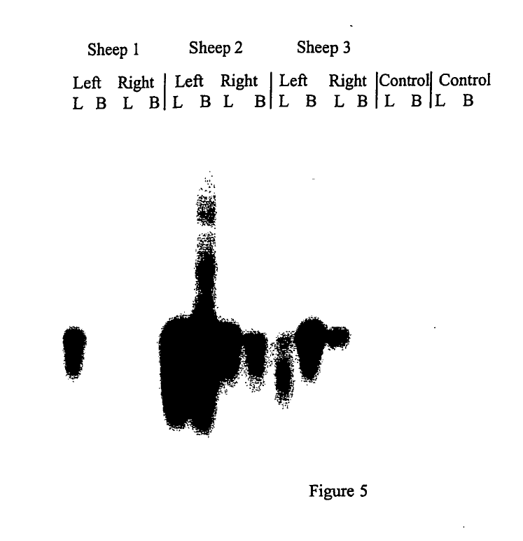

Relatively high levels of galectin-14 mRNA are detected in HDME challenged lung tissue and BAL cells that are associated with eosinophilia (5-38% eosinophils in BAL). In sheep that receive HDME challenge solely to the left lung lobe, there are consistently higher levels of galectin-14 mRNA in the tissue and BAL of that lobe compared to the samples from the right untreated lobe (FIG. 5). The sheep known to have the greatest number of BAL eosinophils (38%) also had the highest levels of galectin-14 mRNA.

As for mammary tissue, galectin-14 mRNA was not detected in whole abomasum RNA after H. contortus larvae infection despite the presence of infiltrating eosinophils. Mammary and abomasal tissue have more structural tissue bulk than lung, and hence the mRNA from infiltrating leukocytes may be diluted in these tissues.

Immunohistology

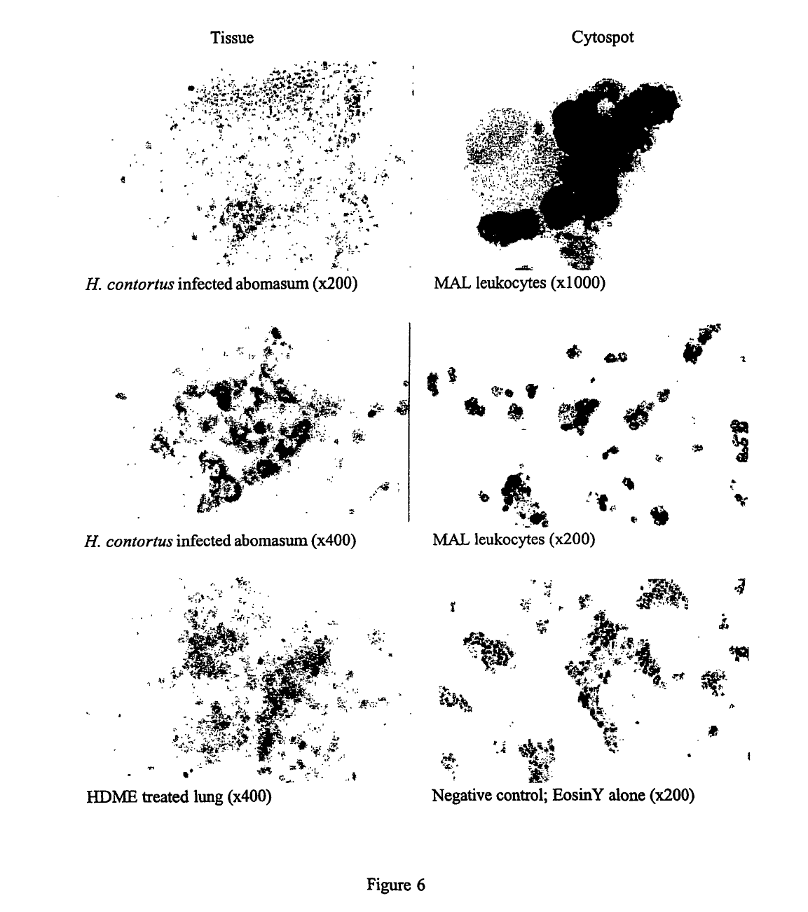

Cytospots of MAL cells and tissue sections containing eosinophils were stained with polyclonal or monoclonal anti-galectin-14 antibodies (FIG. 6). Galectin-14 clearly localised to eosinophils. The staining within the eosinophils was patchy and widespread. It did not appear to localise to the nucleus as found for some other galectins including galectin-10 (Dvorak, A. M., Ishizaka, T., Letourneau, L., Albee, E. A., Mitsui, H. and Ackerman, S. J. (1994) J. Histochem. Cytochem. 42(2):251-263), but appeared to be aggregating under the plasma membrane (FIG. 6).

Western Blot Analysis

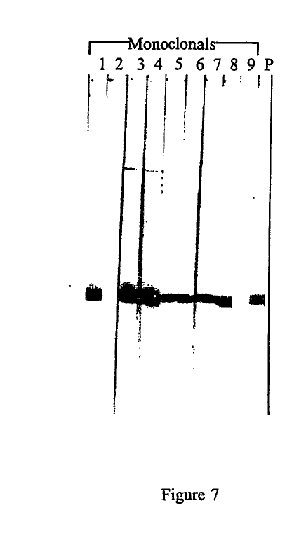

Eosinophil-rich MAL cells solubilized in sample buffer were run on 12.5% SDS-PAGE, transferred to nitrocellulose and probed with polyclonal and monoclonal anti-galectin-14 antibodies (FIG. 7). This clearly detected a protein of similar size to recombinant galectin-14 under both reducing and non-reducing conditions (apparent molecular weight ˜17 kDa). The expected molecular weight of galectin-14 calculated from the predicted amino acid sequence is only slightly larger (18.2 kDa). Additionally, with some mAbs, proteins with higher molecular weight were detected).

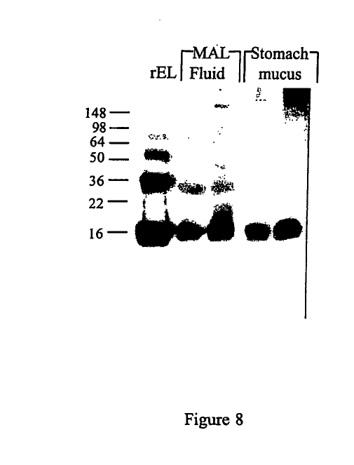

Similarly stomach mucus scrapings collected from H. contortus infected sheep were found to contain a protein of ˜17 kDa that reacts with both polyclonal and monoclonal anti-galectin-14 antibodies (FIG. 8). H. contortus larval infection is known to be associated with eosinophilia, including eosinophils within the mucus layer (Meeusen, E. N. T. and Balic, A. (2000) Parasitol. Today 16:95-101).

Western blot analysis of MAL supernatant (with the cells removed) also indicates that galectin-14 may be secreted (FIG. 8). Like all other galectins galectin-14 does not contain an obvious targeting signal for secretion, and yet both polyclonal and monoclonal anti-galectin-14 antibodies react strongly with a protein of the same size as galectin-14 in MAL fluid samples that are associated with eosinophilia. Some higher molecular weight bands in MAL fluid also reacted with both the polyclonal and monoclonal antibodies. It remains to be determined if the higher molecular weight bands represent cross-reactivity to other proteins, or protein complexes that contain galectin-14; for example homodimerisation of galectin-14, which is known to occur for most other proto-type galectins and is reported to be resistant to reducing agents (Cho, M. and Cummings, R. D. (1995) J. Biol. Chem. 270(10):5198-5206), or galectin-14 bound to the carbohydrate motifs of other proteins.

### EXAMPLE 2

### Isolation and Characterisation of Galectin-14 in Allergen-Primed Animals

Materials and Methods

Collection of Mammary Lavage (MAL) Samples

To induce eosinophil migration into the mammary gland, mature non-lactating Merino ewes were primed every two weeks by intramammary infusions of 1 mg of solubilized house dust mite extract (HDM; Dermatophagoides pteronyssinus; Commonwealth Serum Laboratories Ltd., Melbourne, VIC, Australia), rested for 3-4 weeks and challenged with an intramammary infusion of 1 mg solubilized HDM. MAL was collected 2 days post-HDM challenge by infusion of sterile pyrogen-free saline (PFS; Baxter Healthcare Pty. Ltd, NSW, Australia) followed by ‘milking’ of the gland as described previously (Greenhalgh, et al. (1996) supra; Bischof, R. J. and Meeusen, E. N. T. (2002) Clin. Exp. Allergy, 32:1-8). Cells were pelleted by centrifugation and washed in PFS. The proportion of eosinophils in the leukocyte suspensions, as determined by Giemsa-stained cytospots, varied from 75-90%.

Other sheep received a single intramammary infusion of LPS, and MAL cells were collected at 24 h and 5 days, which results in an initial influx of predominantly neutrophils (24 h) followed by macrophage infiltration at day 5 (Greenhalgh et al. (1996) supra; McDowell, G. H., Lee, C. S. and Lascelles, A. K. (1969) Res. Vet. Sci. 10:13-17).

Collection of Lung Tissue and Bronchoalveolar Lavage (BAL) Samples

4-5 month old parasite free female merino-cross lambs were sensitized by three subcutaneous injections of 50 μg of HDM, solubilized in PFS with aluminum hydroxide as adjuvant (1:1). Sheep that showed a high HDM-specific IgE serum response were challenged 2-3 weeks later with 1 mg of solubilized HDM, in the lower left lung lobe using a fibreoptic bronchoscope (Pentax FG-16×5.5 mm). The right lung lobe of the same sheep was challenged with PFS only as a control. BAL samples were collected from each challenge and control lung site before and 6-48 hours post-challenge, by gently adding and aspirating 5 ml of PFS through the bronchoscope port. Sheep were sacrificed and lung tissue samples collected after the final BAL sample collections (approximately 48 hours post-challenge) for histology. Cells within the BAL were quantified using a Neubauer haemocytometer and eosinophil numbers determined on Giemsa-stained cytospots.

Larger BAL leukocyte populations required for RNA preparation were collected from whole lung lavage of left and right lung lobes by occluding the entrance to one lung lobe with a Foley catheter as described previously (Dunphy, J., Horvath, A., Barcham, G., Balic, A., Bischof, R. and Meeusen, E. (2001) Vet. Immunoo. Immunopathol. 82:153-164). Lung tissue was also collected from each lung lobe for RNA preparation and histology.

Periphleral Blood Leukocytes

Peripheral blood was drawn from the jugular vein of sheep into plastic tubes containing ethylenediaminetetra-acetic acid disodium (EDTA.Na₂; BDH Merck, VIC, Australia). Red blood cells were lysed with TAC (0.17 M Tris/0.16 M NH₄Cl pH 7.2) at 37° C. and the remaining leukocytes washed in PBS, and resuspended in 1% BSA/PBS.

RNA Preparation

Total RNA was purified from 0.1-1 g of tissue or ˜1×10⁸ cells using a standard guanidinium thiocyanate, phenol/chloroform extraction (Chomczynski, P. and Sacchi, N. (1987) Anal. Biochem. 162:156-159).

Low Stringency RT-PCR

cDNA clones differentially expressed by fresh and cultured cells were amplified from eosinophil-rich MAL cells by low stringency RT-PCR using the displayPROFILE kit from Display Systems Biotech (Integrated Sciences, Sydney, Australia) as described in the kit manual (version 2.0). Total RNA from eosinophil-rich MAL cells of nematode challenged sheep (Dunphy, et al. (2001) supra) was used as template. The PCR primer that resulted in amplification of the partial galectin-14 cDNA was DisplayPROBEsEu4 (see Table 3). PCR products of interest were re-amplified and subcloned into pGEM-Teasy (Promega) before being sequenced using the BIG DYE terminator mix (Perkin Elmer Applied Biosystems).

Construction and Screening of a MAL cDNA Library

The SMART cDNA library construction kit (Clontech) was used as instructed by the manufacturer to prepare a cDNA library representing mRNA expressed in an eosinophil-rich leukocyte population as described (Dunphy, et al. (2001) supra). LE392 cells were infected with the pTriplEx2 phage library, and the plaques screened with the original galectin-14 partial RT-PCR cDNA. The ³²P-labeled galectin-14 cDNA probes were generated from the RT-PCR clone using Klenow and the Giga-prime kit (Bresatec, Adelaide, Australia). The hybridization and wash conditions used were the same as for Northern blot analysis (see below). At least 1×10⁶ pfu were used for each primary screen.

Once individual plaques of interest were isolated in tertiary screens, the λTriplEx2 phage was converted into pTriplEx2 plasmid (as instructed in the SMART cDNA manual). The cDNA were then sequenced using the 5′ sequencing primer of pTriplEx2 (supplied by Invitrogen).

Amplification of Galectin-14 cDNA, Containing the Full Putative Coding Region

Two RT-PCR primers were designed within the putative 5′ and 3′ untranslated regions (UTRs) of galectin-14 (G145′UTR and G143′UTR, see Table 3). ˜1.25 μg of MAL cell total RNA was used as a template for reverse transcriptase. 2-10 μl of the RT mix was used as a template for 30 PCR cycles. The PCR used 0.25 μM of each primer, 200 μM of each dNTP and 2.5U of Taq polymerase in a total volume of 100 μl. The 30 PCR cycles utilized a denaturation step of 95° C. for 30 seconds, an annealing temperature of 54° C. for 1 minute, and an extension temperature of 74° C. for 1 minute. An additional denaturation of 5 minutes preceded the 30 cycles, and a prolonged extension of 10 minutes completed the PCR. PCR products were subcloned into pGEM-Teasy and sequenced as above.

Northern Blot Hybridizations

Approximately 10 μg of total RNA was transferred to Hybond N+ membranes (Amersham Pharmacia Biotech) by capillary action. Membranes were prehybridized for 4 h at 42° C. in Church buffer [0.5M sodium phosphate pH7.2/1% BSA/7% SDS/2 mM EDTA]. ³²P-labeled galectin-14 cDNA probes were generated as described above. Probes were hybridized to the membranes in Church buffer overnight at 65° C. The membranes were washed at high stringency in 0.2×SSC/0.1% SDS at 37-42° C.

Production of Recombinant Galectin-14 in E. coli.

To produce recombinant galectin-14 the entire coding region of the mRNA was amplified by RT-PCR and subcloned into the E. coli GST expression vector pGEX-6P-2 (Amersham Pharmacia). The RT-PCR primers used, incorporated 4 nucleotide changes to alter codon usage to that preferred by E. coli (Table 3). The protease deficient E. coli strain BL-21 was used to express recombinant galectin-14 in the form of a GST fusion protein. Expression was induced by addition of 0.1 mM IPTG for 2-3 h at 34° C. The fusion protein was then isolated using a glutathione-sepharose column and cleaved with PreScission protease on the column as instructed by the manufacturer (Amersham Pharmacia). The cleaved and purified galectin-14 recombinant protein contained an additional 5 amino acids at its NH-terminus (remnants of the vectors cleavage and multiple cloning sites). The purity of the protein preparation was confirmed by Coomassie-stained reducing SDS-PAGE and the protein was N-terminally sequenced to confirm that it was in-frame and cleaved appropriately.

Production of Galectin-14 Monoclonal Antibodies

BALB/c mice were given IP injections of ˜5 μg of cleaved and purified recombinant galectin-14 once a month for 3 months, initially in complete Freund's adjuvant and subsequently in incomplete Freunds adjuvant. Spleen cells from immune mice were fused with NS-1 myeloma cells using 50% polyethylene glycol 4000 (Merck, Darmstadt, Germany), and supernatants screened for galectin-14 binding by ELISA. Positive hybridomas were cloned by limiting dilution at least three times before being converted to DM10 media alone. Ascitic fluid was produced by giving pristine primed BALB/c mice an IP injection of 1×10⁷ hybridoma cells. All antibodies used were tested for cross-reactivity to other galectins by testing their affinity to recombinant ovine galectin-11 (Dunphy et al. (2000) supra).

Western Blot Analysis

Proteins from recombinant protein preparations, leukocyte protein preparations, or cell-free MAL and BAL supernatants, were separated by 12.5% SDS-PAGE and transferred to 0.45 μM nitrocellulose membranes (MSI, Melbourne, Australia) by electroblotting at 100V for 1 h. Membranes were incubated with mAb supernatant for 2 h at room temperature, followed by incubation with secondary antibody, horseradish peroxidase-conjugated rabbit anti-mouse Ig (Dako, Carpinteria, Calif.), for at least 1 h at room temperature. Signals were detected using 1.5 mM 3,3′-diaminobenzidine tetrahydrochloride (DAB; Sigma), or ECL (Amersham Pharmacia Biotech).

Immunocytochemistry

For cytospots, 1×10⁵ cells were added to a cytocentrifuge chamber and centrifuged at ˜90 g for five minutes. The slides were dried and fixed in 95% ethanol for 10 min. Endogenous peroxidase was quenched by submerging slides in 1.5% H₂O₂/PBS for 10 min. Slides were incubated with mAb for 1-2 h at room temperature in a humidified chamber, followed by the secondary antibody, for 1 h. Conjugate binding was detected by incubating slides with DAB for 10 min at room temperature. The slides were counterstained with hematoxylin and EosinY.

Tissues were fixed in 10% neutral-buffered formalin and processed to paraffin. Sections were pre-blocked with normal sheep serum (NSS)/PBS for 20 min prior to immunochemical staining as above.

Flow Cytometry

Cells were processed for surface expression of galectin-14 using standard procedures (3,4). For intracellular staining, cells were washed twice in ice-cold PBS then fixed in 4% formaldehyde for 25 min at room temperature. Cells were resuspended in wash buffer (1% BSA/0.05% azide/PBS) and transferred in 50 μl aliquots to a 96-well V-bottomed plate. Following centrifugation, 100 μl of permeabilization buffer, (wash buffer supplemented with 0.5% saponin; Sigma), was added to each well and the cells incubated for 10 min. Primary and secondary antibody incubations were performed in 0.5% saponin at room temperature. Cells were incubated with 50 μl of the primary antibody for 20 min, washed twice with permeabilization buffer, resuspended in 10 μl of 10% NSS/0.1% saponin/PBS and incubated for 10 min, followed by incubation with PE-conjugated sheep anti-mouse Ig (Silenus) for 30 min. Cells were then washed twice with permeabilization buffer, once with wash buffer and resuspended in PBS for analysis on a FACS Calibur® instrument (Becton-Dickinson, Mountain View, USA) using Cellquest software (Becton-Dickinson).

Results

Isolation of a Novel Galectin cDNA

Whilst screening for cDNA clones that were differentially expressed in fresh and cultured eosinophil-rich mammary lavage (MAL) cells, a partial cDNA clone of 325 bp was isolated showing similarity to the potent human eosinophil chemoattractant ecalectin/galectin-9. Northern blot analysis confirmed that this clone was expressed at relatively high levels by the eosinophil-rich leukocyte population. Hence an eosinophil-rich MAL cell cDNA library was screened to isolate the full-length clone (galectin-14).

The entire coding region (including the stop codon) is 489 bp and encodes a predicted protein of 162 amino acids² (FIG. 1). The 5′ untranslated region is only 35 bp and contains a putative binding site for the transcription factor TCF11. The 3′ untranslated region is approximately 260 nucleotides (not including the poly (A) tail) and contains another putative TCF11 binding site as well as a putative signal transducer and activator of transcription (STAT) 5 binding site (MatInspector V2.2, available at http://www.gsf.de/cgi-bin/matsearch.pl).

A PROSITE database search (Nowak, T. P., Haywood, P. L. and Barondes, S. H. (1976) Biochem. Biophys. Res. Commun. 68:650-657) indicates that the predicted protein contains three potential casein kinase II sites, two putative protein kinase C sites and a possible N-glycosylation site (FIG. 1). Hydrophobicity plots (obtained from ProtScale; http://expasy.proteome.org.au/cgi-bin/protscale) also indicate that the protein contains four short hydrophobic regions but is not likely to span a membrane.

Both a nucleotide Blast database search and a protein Blast search (using the predicted amino acid sequence) indicate that the clone encodes a novel galectin (Table 4 and FIG. 2). The nucleotide and predicted protein sequences of galectin-14 demonstrate most identity to ecalectin/galectin-9 (58% and 57% amino acid identity to rat and human galectin-9 respectively; see Table 4).

Galectin-14 contains six of the seven residues (His⁷⁴, Asn⁷⁶, Arg⁷⁸, Asn⁸⁸, Trp⁹⁵, Glu⁹⁸; FIG. 2) reported to be important for sugar recognition and binding by galectin-1 (Bairoch et al. (1997) supra; Hirabayashi, et al. (1994) supra). Additionally, the amino acid alteration detected in one of these seven residues is a conservative change (R to K₁₀₀; FIG. 2). Like many other galectins, galectin-14 contains a Bcl-2 like motif within the CRD.

Eosinophil-Specific Expression of Galectin-14

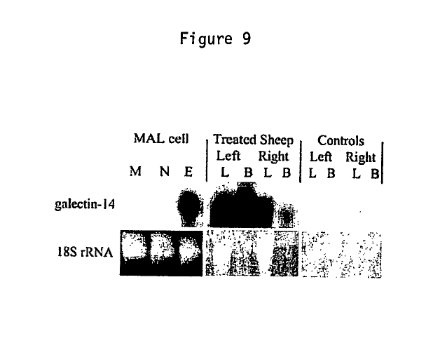

Northern blot analysis detected relatively high levels of galectin-14 mRNA in eosinophil-rich leukocyte populations recovered from the mammary lavage after intramammary infusions of HDM (FIG. 9). Galectin-14 mRNA was not detected in macrophage- or neutrophil-rich MAL leukocyte populations induced by LPS intramammary infusions (FIG. 9), indicating that the gene may be expressed by eosinophils and not other leukocyte populations.

Recombinant galectin-14 was produced as a GST fusion protein in E. coli. To study expression of the galectin-14 protein, galectin-14 mAbs were raised against cleaved and purified recombinant galectin-14. A mAb with high reactivity for galectin-14, but no cross-reactivity with another ovine galectin (OVGAL11; 11) was selected and used to study endogenous galectin-14 protein expression.

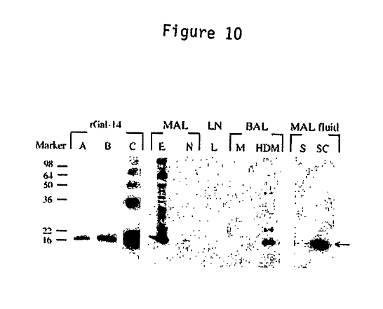

Eosinophil-rich MAL and BAL cells solubilized in sample buffer were run on SDS-PAGE, transferred to nitrocellulose and probed with the galectin-14 mAb (FIG. 10). This clearly detected a protein of similar size to recombinant galectin-14 under both reducing and non-reducing conditions (apparent molecular weight ˜17 kDa). The expected molecular weight of galectin-14 calculated from the predicted amino acid sequence is only slightly larger (18.2 kDa). In concentrated samples or after storage, higher molecular weight bands could often be observed in both recombinant and native samples, probably due to aggregation (FIG. 10). These aggregates did not dissociate even when samples were run of gels under reducing conditions (FIG. 10). Occasionally, higher molecular weight bands were detected by galectin-14 mAb that did not correspond to the predicted mass of oligomers, especially in samples that contain relatively large amounts of monomeric galectin-14 (FIG. 10). These may be the result of post-translational processing of galectin-14 or due to galectin-14 forming stable complexes with other cellular proteins.

In agreement with the Northern blot analysis, Western blots did not, or only weakly, detect galectin-14 in neutrophil- or macrophage-rich cell populations, or in lymph node lymphocytes (FIG. 10). The weak reactions observed in some neutrophil and lymphocyte preparations were probably due to contaminating (1-2%) eosinophils present in these populations detected by the highly sensitive ECL assay, as no staining was observed in these cells on cytospots.

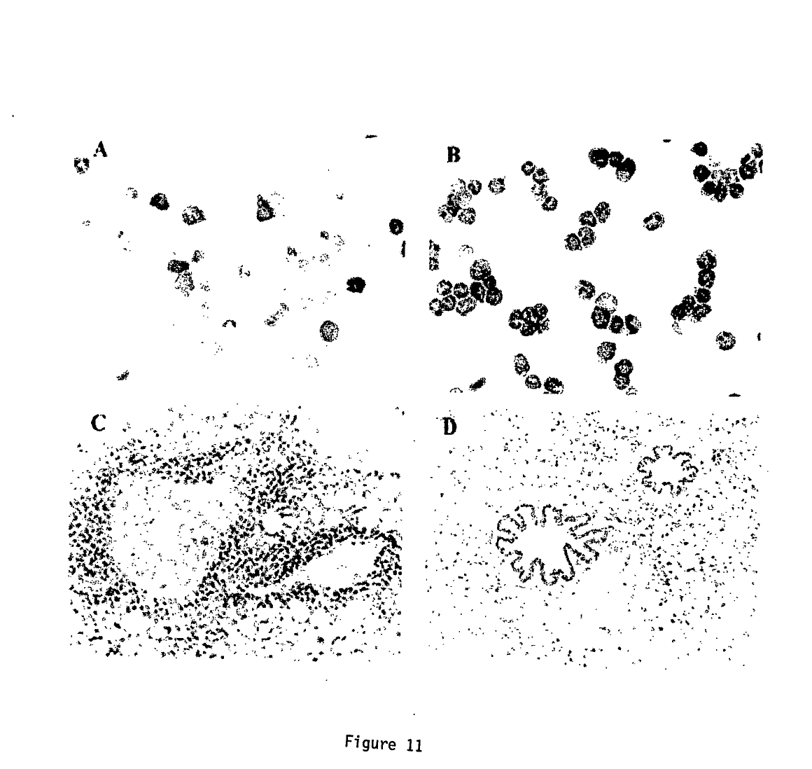

Detailed examination of cytospots prepared from circulating blood cells and eosinophil-rich MAL or BAL cells of HDM sensitized and challenged sheep (FIG. 11A & B), confirmed the localization of galectin-14 to eosinophils, and not neutrophils or lymphocytes. The galectin-14 staining in eosinophils was patchy and widespread within the cytoplasm, with occasional staining of the nuclei, but did not appear to localize to the granules.

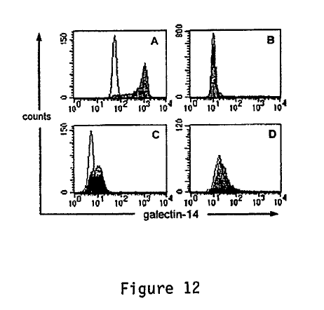

FACS analysis detected strong galectin-14 intracellular staining in more than 95% of eosinophils isolated from mammary lavage after allergen challenge. In contrast, no intracellular staining was detected in neutrophils and macrophages, and only weak non-specific staining in lymphocytes (FIG. 12). The non-specific nature of the absorbence shift in lymphocytes was confirmed by negative staining of lymphocytes in both cytospots and lymph node sections. No galectin-14 surface staining was detected on eosinophils or any other class of leukocytes.

Expression of Galectin-14 in Lung Tissue and its Release into the Lumen of the Lung After Allergen Challenge

To study galectin-14 expression and release in lung tissue a sheep asthma model was developed. Sheep were sensitized to HDM and then challenged with HDM or PFS in the left and right lung lobes respectively. Relatively high levels of galectin-14 mRNA were detected in lung tissue and BAL cells of HDM-sensitized and lung challenged sheep (FIG. 9). There were consistently higher levels of galectin-14 mRNA in the lung tissue and BAL of the left, challenged lung lobe, compared to the samples from the right control lobe (FIG. 9). The level of expression was associated with lung eosinophilia, with the sheep known to have the greatest number of BAL eosinophils (38%) exhibiting the highest levels of galectin-14 mRNA. Weak or no expression was observed in the lungs of control, unchallenged sheep (FIG. 9).

Lung tissue sections of both left and right lung lobes were stained with galectin-14 mAb (FIG. 11C & D). Numerous positive staining eosinophils were present around the bronchioles and blood vessels of the left HDM-challenged lung lobes (FIG. 11C), while the right, saline challenged lungs of the same sheep showed only sporadic eosinophils (FIG. 11D).

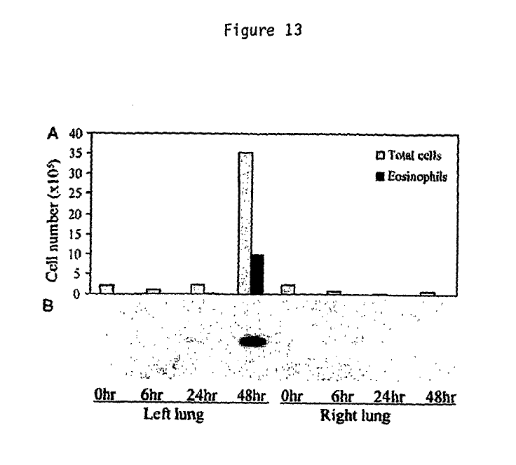

Western blot analysis detected galectin-14 in cell-free MAL fluid (FIG. 10), indicating that galectin-14 may be released into the extracellular environment. The presence of galectin-14 in the MAL fluid was associated with eosinophilia and was only detected in the MAL fluid of sheep challenged with allergen (FIG. 10). The identity of this band to galectin-14 was confirmed by matrix-assisted laser desorption ionization (MALDI) mass spectrometry. To determine if the galectin-14 molecule was also released into the lung, HDM sensitized sheep were challenged in the left and right lung lobes with HDM and PFS respectively, and small BAL samples collected from the challenge sites by gentle deposition and aspiration of PFS at different time points. Western blot analysis of cell-free BAL fluid (FIG. 13) clearly detected the presence of galectin-14 protein in the left lung at 24 and 48 hours after local HDM challenge, while galectin-14 was not detected at any time in the BAL fluid of the right, control lung lobe. The amount of galectin-14 present in the cell free BAL fluid correlated with the numbers of eosinophils present in the lavage, with the time point corresponding to most eosinophil infiltration (48 h) exhibiting the highest level of galectin-14 release in the BAL fluid (FIG. 13). No eosinophils were observed in the right, control lung lobe at any of the time points.

### EXAMPLE 3

### Hemagglutination Assays

Materials and Methods

Hemagglutination Assays

Trypsin-treated, glutaraldehyde fixed rabbit erythrocytes were prepared and tested in an agglutination assay according to the method of Nowak et al. (Nowak et al. (1976) supra). Agglutination assays were performed in 96-well V-shaped microtiter plates with serial 2-fold dilutions of samples in 25 μl of DES (0.15 M NaCl, 2 mM EDTA, 2 mM dithiothreitol), 50 μl of 0.5% (w/v) BSA in 0.15 M NaCl and 25 μl of a 4% suspension of rabbit erythrocytes. The plates were shaken vigorously for 30 s, and agglutination was read after the plates had stood at room temperature for 1 h. Agglutinated erythrocytes formed a “mat” on the bottom of the well. For assessment of the inhibitory effects of saccharides, lactose, galactose, and N-acetyl-glucosamine were serially diluted in 25 μl of 0.15 M NaCl prior to the addition of recombinant protein in DES, 25 μl of 1% (w/v) BSA in 0.15 M NaCl, and 4% erythrocytes in PBS. The minimum concentration of GST-galectin-14 that gave mat formation (0.28 μM) was used in the saccharide inhibition assay. For assessment of the effects of antibody on GST-galectin-14 hemagglutination, ascites and supernatants from monoclonal antibodies and polyclonal rabbit sera all raised to recombinant galectin-14 were serially diluted in 25 μl of 0.15 M NaCl prior to the addition of recombinant protein in DES, 25 μl of 1% (w/v) BSA in 0.15 M NaCl, and 4% erythrocytes in PBS. A different batch of GST-galectin-14, which produced mat formation at a minimum concentration of 0.059 μM, was used at 0.059 μM and 0.042 μM to measure both inhibition and potentiation of agglutination by antibodies.

Laminin Binding Assay

Laminin binding activity was measured according to the method of Mazurek et al. with some modification. Briefly, the wells of a 96-well microtitre plate were coated with laminin from Engelbreth-Holm-Swarm murine sarcoma (Sigma) at 1 μg/100 μl of carbonate coating buffer (15 mM Na₂CO₃, 35 mM NaHCO₃, pH 9.6) per well overnight at 4° C. Non-specific binding sites were blocked with 200 μl of 0.5% Tween 20 in PBS for 1 h. Plates were washed 3 times in 0.05% Tween 20 PBS and incubated with 50 μl serial dilutions of GST-galectin-14 or GST alone, diluted in 0.5% Tween 20 PBS, for 1.5 h at 37° C. Control wells contained 0.3 M lactose. After washing as before, 50 μl rabbit anti-GST horse radish peroxidase (HRP)-conjugated antibody (Sigma) was added at 1/10,000 and the plates incubated for 37° C. for 1 h. Washed plates were developed with 100 μl TMB substrate for 10 min in the dark at room temperature. The reaction was terminated with 100 μl 2 M sulphuric acid and the colour change monitored using a dual wavelength mode plate reader (Bio-tek instruments) at 450 nm, ref 690 nm.

Results

Recombinant Galectin-14 Exhibits Hemagglutination Activity

Fresh cleaved and purified galectin-14 was found to exhibit agglutination activity on rabbit erythrocytes at a minimum concentration of 0.16 μM. However, this activity was rapidly lost upon storage, even in the presence of reducing agents. In contrast, the agglutination activity of recombinant GST-galectin-14 fusion protein did not deteriorate upon storage, and the GST moiety did not appear to interfere with agglutination. Hence GST-galectin-14 was used in subsequent studies.

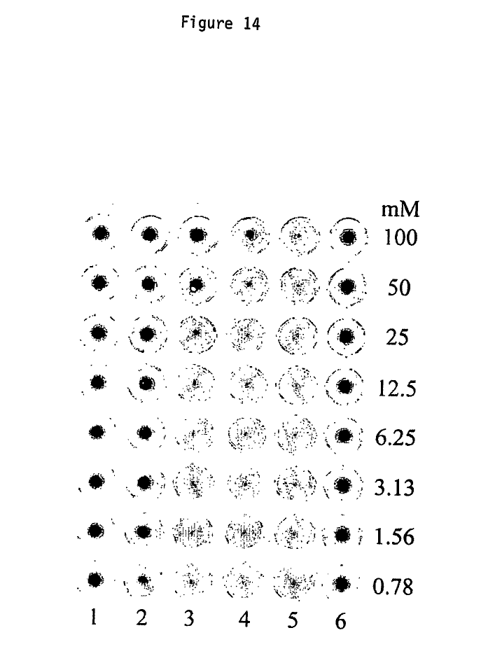

Saccharide binding studies confirmed the agglutination activity of recombinant GST-galectin-14 and demonstrated that this hemagglutination could be inhibited by the addition of sugars (FIG. 14). The presence of 0.28 μM GST-galectin-14 clearly induces hemagglutination, whereas GST alone does not. However, this agglutination is readily inhibited by lactose, with doses as low as 0.78 mM capable of reducing agglutination. The activity could also be inhibited to a lesser extent by galactose and N-acetyl-glucosamine. (Inhibition becoming evident at 12.5 and 50 mM, respectively.)

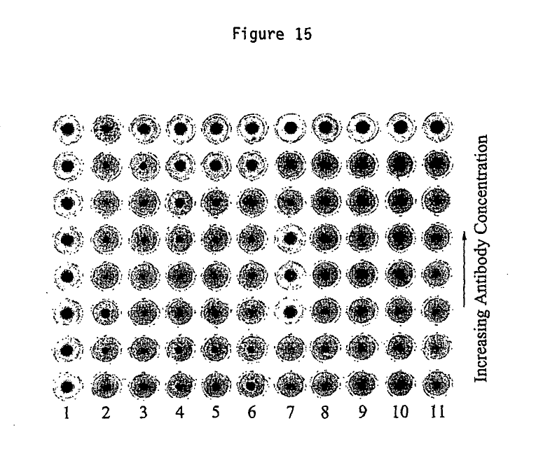

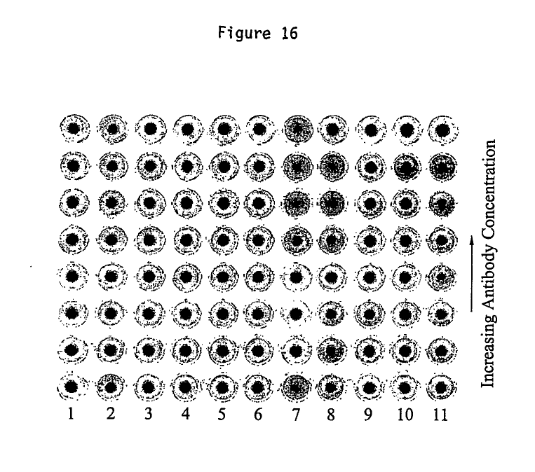

GST-galectin-14 induced hemagglutination could also be inhibited by antibodies raised to recombinant galectin-14 (FIG. 15). All antibodies tested produced some degree of inhibition, however ascites from monoclonal antibody 1.2 (FIG. 15, lane 4) and the polyclonal rabbit sera (FIG. 15, lane 7) appeared to produce the greatest inhibition at the concentrations tested. It is interesting to note that both high and low concentrations of the polyclonal rabbit sera produced inhibition, but not the concentrations in between. In contrast, the polyclonal rabbit sera (FIG. 16, lane 7) and the supernatant of the monoclonal 1.4 (FIG. 16, lane 8) both appeared to potentiate the. agglutination produced by GST-galectin-14, and in the case of the polyclonal sera this was again a biphasic interaction, occurring at both high and low serum concentrations but not in betweeen. No antibody preparation had an effect on hemagglutination in the absence of GST-galectin-14.

Recombinant GST-Galectin-14 Exhibits Laminin Binding Activity

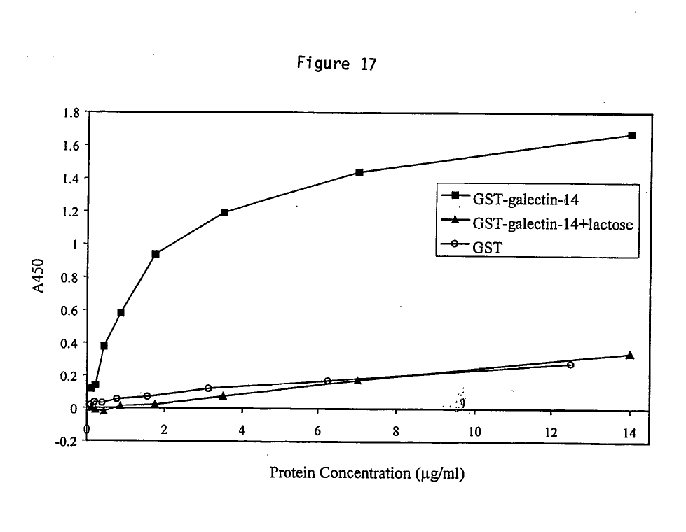

GST-galectin-14 bound to laminin in a dose dependent manner (FIG. 17). This binding was reduced to the level of the control, GST alone, when lactose was added, implying that binding of GST-galectin-14 to laminin is mediated by the carbohydrate recognition domain of galectin-14.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 3

Primers and adapters utilized in low stringency
and conventional RT-PCR

Annealing
temperature

| | | |
|---|---|---|
| Name | Sequence | (° C.) |

| | | | |
|---|---|---|---|
| O-extension primer | <400> 22 | GGTACCGCAGTCTACGAGACCAGT | 55-60 |
| DisplayPROBEsEu4 | <400> 23 | ATGAGTCCTGACCGAAAG | 55-60 |
| G14 5′UTR | <400> 24 | ATTCCTGTTGCAGAAGTCTACCTGGACA | 54 |
| G14 3′UTR | <400> 25 | GAACATCTTCCACACGGTAGGGGT | 54 |
| G14 5′ pGEX | <400> 26 | AGGATCCATGCAGAGCGAAAGTGGTCACGA | 59 |
| G14 3′ pGEX | <400> 27 | CGGCGGCCGCTTAAATCTGGAAGCTGATAT | 59 |

The sequences of adapters and primers used in low stringency or conventional RT-PCR are shown. Annealing temperatures utilized in PCRs are also indicated. The G14 3′UTR primer and G14 3′pGEX primer were used as both RT and downstream PCR primers. Nucleotide substitutions introduced in primers G14 5′pGEX and G14 3′pGEX to alter codon usage to that preferred by E. coli are shown in boldface.

TABLE 4
Amino acid identities between known galectins and galectin-14.

| | | amino acid identity |
|---|---|---|
| Species | Name | (%) |
| Rat | Galectin-9 | 58,00 |
| Human | Galectin-9/ | 57.33 |
| | ecalectin | |
| Mouse | Galectin-9 | 55.33 |
| Mouse | Galectin-6 | 41.45 |
| Human | Galectin-4 | 40.13 |
| Human | Galectin-8 | 38.56 |
| Porcine | Galectin-4 | 38.16 |
| Human | Galectin-7 | 29.93 |
| Rat | GRIFIN | 27.54 |
| Ovine | Galectin-1 | 27.34 |
| Rat | Galectin-5 | 26.85 |
| Human | Galectin-10 | 24.65 |
| Human | Galectin-3 | 24.10 |
| Ovine | Galectin-11 | 22.70 |
| Pig | Galectin-2 | 22.66 |

Amino acid identities between galectin proteins and the predicted protein sequence of galectin-14. These identities were calculated from multiple sequence alignments. Note that the various galectin-9 isoforms all give the same amino acid and nucleotide identity because the putative galectin-14 protein finishes at the end of the N-terminal CRD, and does not cross the linker sequence that varies between these different isoforms.

### BIBLIOGRAPHY

Ackerman, S. J., Corette, S. E., Rosenberg, H. F., Bennett, J. C., Mastrianni, D. M., Nicholson-Weller, A., Weller, P. F., Chin, D. T., and Tennen, D. G. (1993) J. Immunol. 150:456-468

Akahani, S., Nangia-Makker, P., Inohara, H., Kim, H. R. and Raz, A. (1997) Cancer Res. 57:4272-5276

Adams and Colditz, 1991

Ahmed, H., Fink, N. E., Pohl, J. and Vasta, G. R. (1996) J. Biochem. 120:1007-1019

Bairoch, A., Bucher, P. and Hofmann, K. (1997) Nucl. Acids Res. 25:217-221

Balic et al., 2000

Bischof, R. J. and Meeusen, E. N. T. (2002) Clin. Exp. Allergy, 32:1-8

Bonner et al (1973) J. Mol. Biol., 81:123

Carpino et al. (1991)

Cho, M. and Cummings, R. D. (1995) J. Biol. Chem. 270(10):5198-5206

Chomczynski, P. and Sacchi, N. (1987) Anal. Biochem. 162:156-159

Chung et al., 2000

Cooper, D. N. W. and Barondes, S. H. (1990) J. Cell Biol. 110:1681-1691

Cortegano, I., del Pozo, V., Carbada, B., de Andres, B., Gallardo, S., del Amo, A., Arrieta, I., Jurado, A., Palomino, P., Liu, F. T. and Lahoz, C. (1998) J. Immunol. 161:385-389

Douillard and Hoffman, Basic Facts about Hybridomas, in Compendium of Immunology Vol II, ed. by Schwartz, 1981

Dunphy, J. L., Balic, A., Barcham, G. J., Horvath, A. J., Nash, A. D. and Meeusen, E. N. T. (2000) J. Biol. Chem. 275:32106-32113

Dunphy, J., Horvath, A., Barcham, G., Balic, A., Bischof, R. and Meeusen, E. (2001) Vet. Immunoo. Immunopathol. 82:153-164

Dvorak, A. M., Ishizaka, T., Letourneau, L., Albee, E. A., Mitsui, H. and Ackerman, S. J. (1994) J. Histochem. Cytochem. 42(2):251-263

Fourney, R. M., Mlyakoshi, J., Day III, R. S. and Paterson, M. C. (1988) Focus 10(1):5-6

Galfre, G., Howe, S. C., Milstein, C., Butcher, G. W. and Howard, J. C. (1977) Nature 266:550-552

Gardsvoll et al. (1999) J. Biol chem 274(53):37995-38003

Giembycz and Lindsay, 1999

Goodson et al. (1994) Proc. Natl. Acad. Sci. USA 91(15):7129-7133

Greenhalgh, C. J., Jacobs, H. J. and Meeusen, E. N. T. (1996) Immun. Cell Biol. 74:497-503

Hadari, Y. R., Arbel-Goren, R., Levy, Y., Amsterdam, A., Alon, R., Zakut, R., and Zick, Y. (2000) J. Cell Sci. 113:2385-2397

Harlow and Lane 1988

Hirabayashi, J. and Kasai, K. (1994) Glycoconj. J. 11(5):437-442

Hirashima, M. (2000) Int. Arch. Allergy Immunol. 122 (Suppl 1):6-9

Hoyer-Hansen et al. (1997) FEBS Lett 420(1):79-85

Kay, A. B., Barata, L., Meng, Q., Durham, S. R. and Ying, S. (1997) Int. Allergy Immunol. 113:196-199

Kita et al., 1996

Kohler and Milstein, Nature 256: 495-499, 1975; European Journal of Immunology 6: 511-519, 1976

Kuwabara, I. and Liu, F. T. (1996) J. Immunol. 156:3939-3944

Leal-Pinto, E., Wenjing, T., Rappaport, J. Richardson, M., Knorr, B. A. and Abramson, R. G. (1997) J. Biol. Chem. 272:617-625

Liu, 1993

Matsumoto, R., Matsumoto, H., Seki, M., Hata, M., Asano, Y., Kanegasaki, S., Stevens, R. L. and Hirashima, M. (1998) J. Biol. Chem. 273:16976-16984

Matsushita, N., Nishi, N., Seki, M., Matsumoto, R., Kuwabara, I., Liu, F.-T., Hata, Y., Nakamura, T. and Hirashima, M. (2000) J. Biol. Chem. 275:8355-8360

McDowell, G. H., Lee, C. S. and Lascelles, A. K. (1969) Res. Vet. Sci. 10:13-17

Mehul and Hughes, 1997

Meeusen, E. N. T. and Balic, A. (2000) Parasitol. Today 16:95-101

Mehul, B. and Hughes, R. C. (1997) J. Cell Sci. 110:1169-1178

Navaza, 1994

Nowak, T. P., Haywood, P. L. and Barondes, S. H. (1976) Biochem. Biophys. Res. Commun. 68:650-657

Pace et al., 2000

Perillo, N. L., Uittenbogaart, C. H., Nguyen, J. T. and Baum, L. G. (1997) J. Exp. Med. 185:1851-1858

Perillo et al., 1998

Ploug et al. (1994) Biochemistry 33(30):8991-8997

Ploug et al. (1995) Biochemistry 34(39):12524-12534

Ploug et al. (1998) Biochemistry 37(47):16494-16505

Ploug et al. (1998) Biochemistry 37(110):3612-3522

Rabinovich, G. A., Ariel, A., Hershkoviz, R., Hirabayashi, J., Kasai, K.-I. and Lider, O (1999) Immunology 97:100-106

Rabinovich, G. A., Daly, G., Dreja, H., Tailor, H., Riera, C. M., Hirabayashi, J. and Chernajovsky, Y. (1999) J. Exp. Med 190:385-398

Rainbird, M. A., Macmillan, D., and Meeusen, E. N. (1998) Parasite Immunol. 20(2):93-103

Sano, H., Hsu, D. K., Yu, L., Apgar, J. R., Kuwabara, I., Yamanaka, T., Hirashima, M. and Liu, F. T. (2000) J. Immunol. 165:2156-2164

Sato et al., (1993)

Stephens et al, (1992)

Stewart et al. (1985)

Terrett, N. (2000) Drug Discov Today 5(5):211-212

Towbin, H., Stachelin, T. & Gordon, J. (1979) Proc. Natl. Acad. Sci. (USA) 76:4350-4354

Türeci, O., Schmitt, H., Fadle, N., Pfreundschuh, M and Sahin, U (1997) J. Biol. Chem. 272:6416-6422

Vasta, G. R., Quenenberry, M., Ahmed, H. and O'Leary, N. (1999) Dev. Comp. Immunol. 23:401-420

Vespa, g. N., Lewis, L. A., Kozak, K. R., Moran, M., Nguyen, J. R., Baum, L. G. and Miceli, M. C. (1999) J. Immunol. 162:799-806

Wada, J. and Kanwar, Y. S. (1997) J. Biol. Chem. 272:6078-6086

Yamaoka, A., Kuwabara, I., Frigeri, L. G. and Liu, F. T. (1995) J. Immunol. 154:3479-3487

Yang, R., Y., Hsu, D. K. and Liu, F. T. (1996) Proc. Natl. Acad Sci. USA 93:6737-6742

1. An isolated nucleic acid molecule or derivative, homologue or analogue thereof comprising a nucleotide sequence encoding or complementary to a sequence encoding a novel galectin-14 protein or a derivative, homologue or mimetic thereof wherein said galectin-14 comprises one carbohydrate recognition domain.

2. An isolated nucleic acid molecule or derivative, homologue or analogue thereof comprising a nucleotide sequence encoding, or a nucleotide sequence complementary to a nucleotide sequence encoding, an amino acid sequence substantially as set forth in SEQ ID NO:2 or a derivative, homologue or mimetic thereof or having at least about 45% or greater similarity to at least 10 contiguous amino acids in SEQ ID NO:2.

3. An isolated nucleic acid molecule or derivative, homologue or analogue thereof comprising a nucleotide sequence substantially as set forth in SEQ ID NO:1 or a derivative, homologue or analogue thereof or capable of hybridising to SEQ ID NO:1 under low stringency conditions.

4. An isolated nucleic acid molecule according to claim 3 which further encodes an amino acid sequence corresponding to an amino acid sequence set forth in SEQ ID NO:2 or a sequence having at least about 45% similarity to at least 10 contiguous amino acids in SEQ ID NO:2.

5. An isolated nucleic acid molecule according to claim 3 substantially as set forth in SEQ ID NO:1.

6. An isolated novel galectin-14 protein or derivative, homologue, analogue, chemical equivalent or mimetic thereof wherein said galectin-14 comprises one carbohydrate recognition domain.

7. An isolated protein comprising an amino acid sequence substantially as set forth in SEQ ID NO:2 or a derivative, homologue or mimetic thereof or a sequence having at least about 45% similarity to at least 10 contiguous amino acids in SEQ ID NO:2 or a derivative, analogue, chemical equivalent or mimetic of said protein.

8. An isolated protein according to claim 7 encoded by a nucleotide sequence substantially as set forth in SEQ ID NO:1 or a derivative, homologue or analogue thereof or capable of hybridising to SEQ ID NO:1 under low stringency conditions or a derivative, homologue, analogue, chemical equivalent or mimetic of said protein.

9. An isolated protein according to claim 7 substantially as set forth in SEQ ID NO:2.

10. A method of modulating expression of galectin-14, in a mammal, said method comprising contacting the galectin-14 gene with an effective amount of an agent for a time and under conditions sufficient to increase or decrease expression of galectin-14.

11. A method of modulating the functional activity of galectin-14 in a mammal, said method comprising administering to said mammal a modulating effective amount of an agent for a time and under conditions sufficient to increase or decrease galectin-14 activity.

12. The method according to claim 11 wherein said modulation is down-regulation and said agent is an anti-galectin-14 antibody.

13. A method of treating a mammal said method comprising administering to said mammal an effective amount of an agent for a time and under conditions sufficient to modulate the expression of galectin-14 wherein said modulation results in modulation of immune functioning and/or cellular apoptosis.

14. A method of treating a mammal said method comprising administering to said mammal an effective amount of an agent for a time and under conditions sufficient to modulate the activity of galectin-14 wherein said modulation results in modulation of immune functioning and/or cellular apoptosis.

15. The method according to claim 14 wherein said modulation is down-regulation of inflammation and said agent is an anti-galectin-14 antibody.

16. A method of treating a mammal said method comprising administering to said mammal an effective amount of a protein according to claim 6 or a derivative, homologue, analogue, chemical equivalent or mimetic thereof for a time and under conditions sufficient to modulate immune functioning and/or cellular apoptosis.

17. A method of treating a mammal said method comprising administering to said mammal an effective amount of a nucleic acid molecule according to claim 1 or a derivative, homologue, analogue, chemical equivalent or mimetic thereof for a time and under conditions sufficient to modulate immune functioning and/or cellular apoptosis.

18. The method according to claim 14 wherein said immune functioning is inflammation.

19. A method for the treatment and/or prophylaxis of a condition characterised by an aberrant, unwanted or otherwise inappropriate inflammatory response in a mammal said method comprising administering to said mammal an effective amount of an agent for a time and under conditions sufficient to modulate the expression of galectin-14 or sufficient to modulate the activity of galectin-14 wherein up-regulating said expression or activity up-regulates said inflammatory response and down-regulating said expression or activity down-regulates said inflammatory response.

20. A method for the treatment and/or prophylaxis of a condition characterised by an aberrant, unwanted or otherwise inappropriate inflammatory response in a mammal said method comprising administering to said mammal an effective amount of galectin-14 or galectin-14 for a time and under conditions sufficient to up-regulate said inflammatory response.

21. A method of treatment and/or prophylaxis according to claim 19 wherein said condition is an allergic condition and its inflammatory response is a Th2 type inflammatory response which is down-regulated by down-regulation of galectin-14 or functional activity or galectin-14 expression.

22. The method according to claim 20 wherein said agent is an anti-galectin-14 antibody.

23. A method for the treatment and/or prophylaxis of a condition characterised by aberrant, unwanted or otherwise inappropriate cellular apoptosis in a mammal said method comprising administering to said mammal an effective amount of an agent for a time and under conditions sufficient to modulate the expression of galectin-14 or sufficient to modulate the activity of galectin-14 wherein up-regulating said expression or activity down-regulates said apoptosis and down regulating said expression or activity up-regulates said apoptosis.

24. A method for the treatment and/or prophylaxis of a condition characterised by aberrant, unwanted or otherwise inappropriate cellular apoptosis in a manual said method comprising administering to said mammal an effective amount of galectin-14 or galectin-14 for a time and under conditions sufficient to down-regulate said apoptosis.

25. Use of an agent capable of modulating the expression galectin-14 or a derivative, homologue, analogue, chemical equivalent or mimetic thereof in the manufacture of a medicament for the modulation of immune functioning and/or cellular apoptosis.

26. Use of an agent capable of modulating the activity of galectin-14 or a derivative, homologue, analogue, chemical equivalent or mimetic thereof in the manufacture of a medicament for the modulation of immune functioning and/or cellular apoptosis.

27. Use according to claim 26 wherein said immune functioning is an inflammatory response.

28. Use according to claim 26 wherein said modulation is down-regulation of inflammation and said agent is an anti-galectin-14 antibody.

29. Use of galectin-14 or galectin-14 or a derivative, homologue, analogue, chemical equivalent or mimetic thereof in the manufacture of a medicament for the treatment of a condition characterised by aberrant, unwanted or otherwise inappropriate inflammatory response and/or cellular apoptosis.

30. An agent for use in modulating galectin-14 activity or a derivative, analogue chemical equivalent or mimetic thereof wherein modulating said galectin-14 activity modulates immune, functioning and/or cellular apoptosis.

31. An agent according to claim 30 wherein said modulation is down-regulation of inflammation and said agent is an anti-galectin-14 antibody.

32. An agent for use in modulating galectin-14 expression or a derivative, homologue, analogue, chemical equivalent or mimetic thereof wherein modulating expression of said galectin-14 modulates immune functioning and/or cellular apoptosis.

33. An agent according to claim 30 wherein said immune functioning is an inflammatory response.

34. Galeotin-14 or galectin-14 or a derivative, homologue, analogue, chemical equivalent or mimetic thereof for use in modulating immune functioning and/or cellular apoptosis.

35. A pharmaceutical composition comprising galectin-14, galectin-14 or an agent capable of modulating galectin-14 or galectin-14 or derivative, homologue, analogue, chemical equivalent or mimetic thereof together with one or more pharmaceutically acceptable carriers and/or diluents.

36. The pharmaceutical composition according to claim 35 wherein said agent is an anti-galectin-14 antibody.

37. An isolated antibody directed to the protein according to claim 6.

38. An isolated antibody directed to the nucleic acid molecule according to claim 1.

39. An isolated antibody according to claim 37 wherein said antibody is derived from hybridoma clone 1.2 or a derivative, homologue, analogue, chemical equivalent or mimetic of said antibody.

40. The antibody according to claim 37 wherein said antibody is a monoclonal antibody.

41. The antibody according to claim 31 wherein said antibody is a polyclonal antibody.

42. A method for detecting an agent capable of modulating the functioning of galectin-14 or its functional equivalent or derivative thereof or galectin-14 expression said method comprising contacting a cell or extract thereof containing said galectin-14 or its functional equivalent or derivative or galectin-14 with a putative agent and detecting an altered expression phenotype associated with said galectin-14 or its functional equivalent or derivative or galectin-14.

43. A method for detecting an agent capable of binding or otherwise associated with a galectin-14 or galectin-14 binding site or functional equivalent or derivative of said galectin-14 or galectin-14 said method comprising contacting a cell containing said galectin-14 or galectin-14 binding site or functional equivalent or derivative thereof with a putative agent and detecting an altered expression phenotype associated with modulation of the function of galectin-14 or galectin-14 or its functional equivalent or derivative thereof.

44. A method for analyzing, designing and/or modifying an agent capable of interacting with the carbohydrate, protein kinase C and/or casein kinase II binding site of galectin-14 or derivative thereof and modulating at least one functional activity associated with said galectin-14 said method comprising contacting said galectin-14 or derivative thereof with a putative agent and assessing the degree of interactive complementarity of said agent with said binding site.

45. The method according to claim 43 wherein said binding site is defined by one or more of:
(i) the amino acids H⁷⁴, N⁷⁶, R⁷⁸, N⁸⁸, W⁹⁵ and E⁹⁸ of SEQ ID NO:1;
(ii) the amino acid sequence SGHE (position numbers 5-8 of SEQ ID NO:1), STDE (position numbers 51-54 of SEQ ID NO:1), or SLFE (position numbers 110-113 of SEQ ID NO:1);
(iii) the amino acid sequence TGR (position numbers 30-32 of SEQ ID NO:1) or SFK (position numbers 122-124 of SEQ ID NO:1).

46. Agents identified utilising method of claim 43.

47. A method of diagnosing or monitoring a mammalian disease condition said method comprising screening for galectin-14 or galecetin-14 in a biological sample isolated from said mammal.

48. The method according to claim 13 wherein said modulation is down-regulation of inflammation and said agent is an anti-galectin-14 antibody.

49. The method according to claims 19 wherein said agent is an anti-galectin-14 antibody.

50. Use according to claim 25 wherein said immune functioning is an inflammatory response.
